(19) United States
(12) Patent Application Publication
(10) Pub. No.: US 2026/0092282 A1
(43) Pub. Date: Apr. 2, 2026

(54) DNA-RNA HETERODUPLEX TARGETING ANDROGEN RECEPTOR

(71) Applicant: SiRNAgen Therapeutics Incorporated, Boston, MA (US)

(72) Inventors: Han-Oh PARK, Sechong-si (KR); June Young PARK, Boston, MA (US); Sung Il YUn, Sechong-si (KR); Sang Kyu LEE, Daejeon (KR); Woorim CHOI, Daejeon (KR)

(21) Appl. No.: 19/479,629

(22) PCT Filed: Mar. 29, 2024
(86) PCT No.: PCT/US2024/022300

§ 371 Date: Oct. 28, 2025

Related U.S. Application Data
(60) Provisional application No. 63/492,794, filed on Mar. 29, 2023.

(51) Int. Cl. C12N 15/113; A61K 8/60

(52) U.S. Cl. CPC C12N 15/1138; A61K 8/606; C12N 2310/14; C12N 2310/315; C12N 2310/3515; C12N 2320/32

(57) ABSTRACT

The present disclosure provides a DNA-RNA heteroduplex targeting and inhibiting expression of androgen receptor and its composition. The DNA-RNA heteroduplex and the composition can be used to inhibit a symptom associated with expression of androgen receptor. In particular, the method of using the DNA-RNA heteroduplex and the composition for hair improvement, increase of hair growth, or decrease of hair loss are provided.

### 1. CROSS-REFERENCE TO RELATED APPLICATIONS

This application claims the benefit of U.S. Provisional Application No. 63/492,794 filed on Mar. 29, 2023, which is hereby incorporated by reference in its entirety.

### 2. SEQUENCE LISTING

This application contains a Sequence Listing which has been submitted electronically in XML format and is hereby incorporated by reference in its entirety. Said XML copy created on XX, is named 41346-54988-SEQLIST.xml and is XX bytes in size.

### 3. BACKGROUND OF THE INVENTION

Androgenetic alopecia (AGA), commonly known as male pattern hair loss (MPHL) in men and female pattern hair loss (FPHL) in women, is the most common type of progressive hair loss. Androgens, male hormones, are one of the causes of AGA. Although the relationship between FPHL and androgen is unclear, FPHL is accompanied by hair follicle miniaturization and hair thinning, similar to MPHL. The pathogenesis of both diseases is not yet completely understood, but its incidence has increased in recent years.

Androgen and AR signaling plays an essential role in regulating the hair cycle and skin pathogenesis, including in AGA. Endogenous androgens include testosterone and dihydrotestosterone (DHT). DHT is a more potent androgen synthesized from testosterone by 5-α reductase and exhibits about 10-fold higher binding affinity with the androgen receptor (AR). DHT and AR levels are elevated in patients with AGA, and it has been reported that DHT-AR signaling is closely related to AGA pathogenesis. Finasteride and dutasteride, which were developed as 5-α reductase inhibitors, have been approved by the FDA and are being used as the main treatments for AGA. However, these drugs have several side effects, such as a decrease in libido via decreases in DHT.

More recently, siRNA nanoparticles (SAMiRNAs) targeting AR mRNA regions were developed to lower the levels of AR mRNA and protein in DPCs and hair follicles as described in PCT/KR2019/015723, which is incorporated by reference in its entirety. Their effects on hair growth were tested in a clinical study, where application of the SAMiRNA for 24 weeks showed efficacy similar to that of finasteride without side effects. See Yun, S I., Lee, S K., Goh, E A. et al. Weekly treatment with SAMiRNA targeting AR mRNA ameliorated androgenetic alopecia. Sci Rep 12, 1607 (2022).

These studies suggest that the SAMiRNA can be a potential treatment for AGA. There is a need to develop a formulation of the SAMiRNA for safe and effective delivery and a long-term and stable storage of the SAMiRNA. Further, improving safety, quality and efficacy of the drug compound, SAMiRNA, and its use schedule are desired for wide use and commercialization of the formulation.

### 4. SUMMARY OF THE INVENTION

The present disclosure provides a DNA-RNA heteroduplex, comprising: a DNA strand having the sequence of SEQ ID NO: 1 or 3, an RNA strand having the sequence of SEQ ID NO: 2 or 4, or a modified RNA strand comprising the sequence of SEQ ID NO: 2 or 4 with one or more modifications, stearyldisulfidehexyl (C6-S—S—C18) covalently attached to 5′-end of the DNA strand; and hexaethylene glycol (HEG) or polyethylene glycol (PEG) covalently attached to 3′-end of the DNA strand, wherein the DNA strand and the RNA strand or the modified RNA strand form a DNA-RNA hybrid.

In some embodiments, the DNA strand has the sequence of SEQ ID NO: 1 and the RNA strand has the sequence of SEQ ID NO: 2. In some embodiments, the DNA strand has the sequence of SEQ ID NO: 3 and the RNA strand has the sequence of SEQ ID NO: 4.

In some embodiments, the DNA-RNA heteroduplex comprises HEG, wherein the HEG is hexaethyleneglycol-(—PO3-hexaethyleneglycol)₃.

In some embodiments, the DNA-RNA heteroduplex comprises the modified RNA strand, wherein the modified RNA strand comprises the sequence of SEQ ID NO: 2 or 4 with one or more phosphorothioate modifications.

In some embodiments, the modified RNA strand comprises the sequence of SEQ ID NO: 2 and each nucleotide of UCU at 3′-end of SEQ ID NO: 2 is a phosphorothioated nucleotide.

The present disclosure also provides a composition for hair improvement comprising the DNA-RNA heteroduplex disclosed herein. In some embodiments, the composition further comprises one or more humectants selected from betaine and butylene glycol. In some embodiments, the composition further comprises one or more hair conditioning or soothing ingredients selected from panthenol, aminopropanol, biotin, niacinamide, and menthol. In some embodiments, the composition further comprises one or more buffering ingredients selected from citric acid and sodium citrate.

In some embodiments, 0.1-2% by weight of the composition is the DNA-RNA heteroduplex. In some embodiments, 0.25-1% by weight of the composition is the DNA-RNA heteroduplex. In some embodiments, 0.4-0.6% by weight of the composition is the DNA-RNA heteroduplex. In some embodiments, 0.4%, 0.45%, 0.5%, 0.55%, or 0.6% by weight of the composition is the DNA-RNA heteroduplex. In some embodiments, 1%, 1.25%, 1.5%, 1.75% or 2% by weight of the composition is the DNA-RNA heteroduplex.

In some embodiments, 1-20 mg/ml of the composition is the DNA-RNA heteroduplex. In some embodiments, 2.5-10 mg/ml of the composition is the DNA-RNA heteroduplex. In some embodiments, 4-6 mg/ml of the composition is the DNA-RNA heteroduplex. In some embodiments, 4 mg/ml, 4.5 mg/ml, 5 mg/ml, 5.5 mg/ml, or 6 mg/ml of the composition is the DNA-RNA heteroduplex. In some embodiments, 10 mg/ml, 12.5 mg/ml, 1.5 mg/ml, 1.75 mg/ml, or 2 mg/ml of the composition is the DNA-RNA heteroduplex.

In some embodiments, the composition comprises denatured ethanol, D-panthenol, betaine, biotin, niacinamide, citric acid, menthol crystals, trisodium citrate dihydrate, and 1,3-butylene glycol. In some embodiments, the composition comprises 30-35% of phosphate buffered saline, 0.4-0.6% of the DNA-RNA heteroduplex, 45-50% of water, 14-15% of ethanol, 0.19-0.2% of panthenol, 1% of niacinamide, 0.02% of citric acid, 0.05% of menthol, 0.03% of sodium citrate, 3% of butylene glycol and less than 0.01% of aminopropanol. In some embodiments, the composition comprises 32.8% of phosphate buffered saline, 0.5-0.6% of the DNA-RNA heteroduplex, 46.3% of water, 14-15% of ethanol, 0.19-0.2% of panthenol, 1% of niacinamide, 0.02% of citric acid, 0.05% of menthol, 0.03% of sodium citrate, 3% of butylene glycol and less than 0.01% of aminopropanol.

In some embodiments, the composition is in the form of ointment, paste, gel, jelly, serum, aerosol spray, non-aerosol spray, foam, cream, lotion, solution, or suspension formulation.

In some embodiments, the composition has a pH between 5 and 7.5. In some embodiments, the composition has a pH between 5.5 and 6.5, between 6 and 6.5 or between 7 and 7.5. In some embodiments, the composition has a pH 6.29 or 7.02.

In some embodiments, the DNA-RNA heteroduplex forms a nanoparticle.

In some embodiments, the nanoparticle has a mean diameter between 50 and 200 nm. In some embodiments, the nanoparticle has a mean diameter between 50 and 150 nm, between 75 and 125 nm, between 80 and 120 nm, between 90 and 110 nm, or between 100 and 110 nm.

The present disclosure also provides a method of inhibiting androgen receptor expression, comprising applying the DNA-RNA heteroduplex or the composition disclosed herein to a subject.

In some embodiments, the DNA-RNA heteroduplex or the composition is applied topically. In some embodiments, the DNA-RNA heteroduplex or the composition is applied for hair improvement in the subject. In some embodiments, the DNA-RNA heteroduplex or the composition is applied on the scalp of the subject.

In some embodiments, the DNA-RNA heteroduplex or the composition is applied once a week, once per two weeks or once per four weeks. In some embodiments, the DNA-RNA heteroduplex or the composition is applied for at least four months or at least six months. In some embodiments, the DNA-RNA heteroduplex or the composition is applied once a week for at least four months. In some embodiments, the DNA-RNA heteroduplex or the composition is applied once a week for four months.

In some embodiments, the method comprises applying the DNA-RNA heteroduplex or the composition once a week for four months, and applying the DNA-RNA heteroduplex or the composition less than once a week after the four months. In some embodiments, the method comprises applying the DNA-RNA heteroduplex or the composition once a week for four months, and after the four months applying the DNA-RNA heteroduplex or the composition once per 2 weeks, once per 3 weeks or once per 4 weeks.

In some embodiments, the method comprises applying the DNA-RNA heteroduplex or the composition once a week for four months, and after the four months applying the composition once per 1 month, once per 2 months, once per 3 months, once per 4 months, once per 5 months, once per 6 months, or less.

In some embodiments, the method comprises applying the DNA-RNA heteroduplex or the composition once a week for four months, and after the four months discontinuing application of the composition.

In some embodiments, the subject has alopecia. In some embodiments, the subject has moderate androgenetic alopecia. In some embodiments, the subject has Norwood scale stadium at least III or more increased hair loss. In some embodiments, the subject has Ludwig scale stadium at least I-3 or more increased hair loss.

In some embodiments, the composition applied to the subject comprises the DNA-RNA heteroduplex at 1-20 mg/ml. In some embodiments, the composition applied to the subject comprises the DNA-RNA heteroduplex at 2.5-10 mg/ml. In some embodiments, the composition applied to the subject comprises the DNA-RNA heteroduplex at 4 mg/ml, 4.5 mg/ml, 5 mg/ml, 5.5 mg/ml, or 6 mg/ml. In some embodiments, the composition applied to the subject comprises the DNA-RNA heteroduplex at 4.5 mg/ml, 5 mg/ml, or 5.5 mg/ml.

In some embodiments, the method comprises applying 1-100 mg of the DNA-RNA heteroduplex once a week. In some embodiments, the method comprises applying 1-30 mg of the DNA-RNA heteroduplex once a week. In some embodiments, the method comprises applying 5-10 mg of the DNA strand or the RNA strand once a week.

In some embodiments, the method comprises applying 1-10 mg of the DNA strand or the RNA strand once a week. In some embodiments, the method comprises applying 1-100 mg of the DNA-RNA heteroduplex per area scalp (cm²) once a week. In some embodiments, the method comprises applying 1-50 mg of the DNA-RNA heteroduplex per area scalp (cm²) once a week. In some embodiments, the method comprises applying 5-20 mg of the DNA-RNA heteroduplex per area scalp (cm²) once a week. In some embodiments, the method comprises applying 8-10 mg of the DNA-RNA heteroduplex per area scalp (cm²) once a week. In some embodiments, the method comprises applying 0.5-5 ml of the DNA-RNA heteroduplex per area scalp (cm²) once a week. In some embodiments, the method comprises applying 1-2 ml of the DNA-RNA heteroduplex per area scalp (cm²) once a week. In some embodiments, the method comprises applying 1-1.5 ml of the DNA-RNA heteroduplex per area scalp (cm²) once a week.

### 5. BRIEF DESCRIPTION OF THE SEVERAL VIEWS OF THE DRAWINGS

These and other features, aspects, and advantages of the present invention will become better understood with regard to the following description, and accompanying drawings, where:

FIGS. 1A-1C. Screening of potent SAMiRNAs targeting human AR. (FIG. 1A) Schematic of SAMiRNA nanoparticles. (FIG. 1B and FIG. 1C) LNCap cells were treated with PBS or 14 SAMiRNA candidates for 48 hrs. Total RNA extracts were subjected to quantitative polymerase chain reaction (qPCR) assays to evaluate AR knockdown efficacy, and AR expression was normalized to the expression of the ribosomal protein lateral stalk subunit P0 (RPLP0) gene (FIG. 1B). Whole-cell lysates were subjected to immunoblot analysis to compare the protein levels of AR and GAPDH. The intensity of the AR and GAPDH bands was quantified by ImageJ software, and AR protein expression was normalized to glyceraldehyde-3-phosphate dehydrogenase (GAPDH) values (FIG. 1C, lower panel). Statistical significance was assessed by two-sided Student's/test, *** p<0.001.

FIG. 2A and FIG. 2B show silencing effects of AR in LNCap cells analyzed for each SAMiRNA candidate by reverse transcription-quantitative polymerase chain reaction (RT-qPCR). Fourteen SAMiRNA candidates were selected based on their knockdown efficiency (>50% AR silencing efficacy).

FIGS. 3A-3D. The efficacy of SAMiRNA-AR68 in reducing AR expression in HFDP cells. (FIG. 3A and FIG. 3B) Human follicle dermal papilla (HFPD) cells were treated with PBS, SAMiRNA-AR68, or SAMiRNA-AR109 for 48 hrs. Total RNA extracts were subjected to RT-qPCR assays, and AR expression was normalized to that of the RPLP0 gene. (FIG. 3A). Whole-cell lysates were subjected to immunoblot analysis (FIG. 3B, left panel). The intensity of the AR and GAPDH bands was quantified by ImageJ software, and AR protein expression was normalized to GAPDH values (FIG. 3B, right panel). (FIG. 3C) HFDP cells were treated with PBS, SAMiRNA control, or the indicated doses of SAMiRNA-AR68 for 48 hrs. Total RNA extracts were subjected to qPCR assays, and AR expression was normalized to that of the RPLP0 gene. (FIG. 3D) HFDP cells were treated with PBS, SAMiRNA control, or 10 μM SAMiRNA-AR68 for 48 hrs. Whole-cell lysates were subjected to enzyme-linked immunosorbent assays (ELISAs) to measure AR protein. Statistical significance was assessed by two-sided Student's/test, ** p<0.01 and *** p<0.001.

FIGS. 4A-4C. AR silencing efficacy of SAMiRNA-AR68 in human hair follicles. (FIG. 4A) Plucked human hair follicles were treated with PBS or 10 μM FAM-labeled SAMIRNA-AR68 in culture medium for 24 hrs. Hair follicles were subjected to immunofluorescence (IF) analysis and counterstained with 4′,6-diamidino-2-phenylindole (DAPI). Scale bars=100 μm. (FIG. 4B) Plucked human hair follicles were treated with PBS or 10 μM SAMiRNA-AR68 for 48 hrs. Total RNA extracts from hair bulbs were subjected to qPCR assays, and AR expression was normalized to that of the RPLP0 gene. (FIG. 4C) Plucked human hair follicles were treated with PBS or 10 μM SAMiRNA-AR68 for 48 hrs. IF analysis of the plucked vertex hair follicle section incubated with an anti-AR antibody and counterstained with DAPI. Scale bar=100 μm. Levels of AR protein were measured by the mean fluorescence intensity using ZEN software. Data are shown as the mean value±SD (n=8 hairs/group) normalized for DAPI intensity. Statistical significance was assessed by two-sided Student's/test, *p<0.05 and ** p<0.01.

FIGS. 5A and 5B. The safety of SAMiRNA-AR68. (FIG. 5A) HFDP and HaCaT cells were treated with PBS or the indicated dose of SAMiRNA-AR68 for 72 hrs. WST-1 assays were performed to measure cell viability. (FIG. 5B) Human PBMCs were treated with 20 μg/ml concanavalin A (ConA) as a positive control, PBS, or the indicated doses of SAMIRNA-AR68 for 6 hrs. Total RNA extracts were subjected to qPCR assays to evaluate the expression of proinflammatory cytokines, including IL-1β, IL-6, INF-γ and TNF-α, normalized to that of the ribosomal protein L13A (RPL13A) gene.

FIG. 6A provides the average size of the AR68 nanoparticles measured in the formulation after storage for 6 months. The average size was approximately 99.2±5.1 nm (22° C., 55%±5 humidity) and 105.0±2.5 nm (40° C., 75%±5 humidity). FIG. 6B shows AR knockdown by RT-qPCR using AR68 samples stored for 6 months under the indicated conditions.

FIG. 7 is a flowchart summarizing the clinical trial I study described in Section 6.5.

FIGS. 8A and 8B. Images of the change in hair loss following 24 consecutive weeks of application of SAMiRNA-AR68 at 0.5 mg/ml and placebo products. (FIG. 8A) Representative photographs of the forehead hairline and vertex of the two groups at baseline and 8, 16 and 24 weeks after 0.5 mg/ml AR68 and placebo treatment three times per week. The phototrichogram analysis was performed at baseline and at 16 and 24 weeks. (FIG. 8B) Representative graph of the percentage of total hair count increments at 16 and 24 weeks compared to baseline.

FIG. 9 is a flowchart summarizing the clinical trial II study described in Section 6.6.

FIGS. 10A and 10B. Images of the change in hair loss following 24 consecutive weeks of application of SAMiRNA-AR68 at 5 mg/ml and placebo products. (FIG. 10A) Representative photographs of the forehead hairline and vertex of the two groups at baseline and 8, 16 and 24 weeks after 5 mg/ml AR68 and placebo treatment once a week. The phototrichogram analysis was performed at baseline and at 16 and 24 weeks. (FIG. 10B) Representative graph of the percentage of total hair count increments at 16 and 24 weeks compared to baseline.

FIG. 11 provides an exemplary device for packaging and delivering SAMiRNA formulation. It is a piston-type bottle with a silicon adaptor for massage.

FIG. 12 are graphs summarizing demographics of subjects tested in the clinical trial III study described in Section 6.8. The pie chart (left) shows degrees of skin loss of the subjects, and the bar chart (right) shows their age distribution.

FIG. 13A shows the ratio of anagen hair (%) calculated in the placebo and the treatment group (weekly, 2/month, 1/month) before applying SAMiRNA-AR68 (Start) and after treatment with SAMiRNA-AR68 for 6 months (6 mo). FIG. 13B shows the changes in anagen hair percentages after treatment with SAMiRNA-AR68 for 6 months compared to the percentages before the treatment.

FIG. 14A provides mean changes in Anagen Hair (Change in %) in male subjects treated with placebo or SAMiRNA-AR68 for 6 months. FIG. 14B provides mean changes in Anagen Hair (Change in %) in female subjects treated with placebo or SAMiRNA-AR68 for 6 months.

FIG. 15 shows the number of out fallen hair (count) counted in the placebo and the treatment group (weekly, 2/month, 1/month) before applying SAMiRNA-AR68 (Start) and after treatment with SAMiRNA-AR68 for 4 or 6 months.

FIG. 16 shows mean changes in the out fallen hair (Change in %) in subjects treated with placebo or SAMiRNA-AR68 after 4 months.

FIG. 17A shows mean changes in the out fallen hair (Change in %) in the male subjects treated with placebo or SAMiRNA-AR68 after 4 months. FIG. 17B shows mean changes in the out fallen hair (Change in %) in the female subjects treated with placebo or SAMIRNA-AR68 for 4 months.

FIG. 18 shows mean changes in the out fallen hair (Change in %) in the subjects treated with placebo or SAMiRNA-AR68 weekly (4/month), biweekly (2/month) or monthly (1/month) for 4 months or 6 months.

FIG. 19A and FIG. 19B show the density of terminal hair measured as 1/cm² (FIG. 19A) or % (FIG. 19B) in different dosing frequency groups-weekly (4/month), biweekly (2/month) and monthly (1/month), before or 4 or 6 months after treatment with placebo or SAMiRNA-AR68.

FIG. 20 shows the density of vellus hair measured as 1/cm² in different dosing frequency groups-weekly (4/month), biweekly (2/month) and monthly (1/month), before or 4 or 6 months after treatment with placebo or SAMiRNA-AR68.

FIG. 21 shows the hair thickness measured as mean μm in different dosing frequency groups-weekly (4/month), biweekly (2/month) and monthly (1/month), before or 4 or 6 months after treatment with placebo or SAMiRNA-AR68.

FIG. 22 provides representative photographs of the forehead hairline and vertex of different dosing frequency groups—weekly (4/month), biweekly (2/month) and monthly (1/month), before or after treatment with SAMiRNA-AR68.

FIG. 23 provides inhibitory effects on AR mRNA expression in human follicle dermal papilla cells analyzed for SAMiRNA-AR68 or its variant with or without phosphorothioate modifications by reverse transcription-quantitative polymerase chain reaction (RT-qPCR).

FIG. 24 provides half-maximal inhibitory concentration (IC50) of SAMiRNA-AR68 or its variants on AR mRNA expression.

FIG. 25A is a fluorescent image of the mouse skin treated with fluorescein (Cy-5)-labeled SAMiRNA-SRN-021. It shows hypodermal delivery of SAMiRNA-SRN-021 through appendageal (intercellular) penetration. FIG. 25B shows inhibition of AR mRNA expression in the skin (about 50% knockdown) in the skin tissue treated with SAMiRNA-SRN-021.

FIG. 26A shows detection of SAMiRNA by RT-qPCR amplification using stem-loop primers as represented by Ct (cycle threshold). There was no significant difference between the control group and the group topically applied with SAMiRNA-SRN-021. FIG. 26B shows AR mRNA expression measured in the kidney in the mice treated with placebo or SAMIRNA-SRN-021. There was no significant difference between the control group and the group topically applied with SAMiRNA-SRN-021.

### 6. DETAILED DESCRIPTION OF THE INVENTION

### 6.1. Definitions

The term “hair improvement” is used to refer to prevention of hair loss and/or increase of hair growth, but not limited thereto. Hair improvement, as used herein, can be improvement of hair health, strength, and/or thickness.

### 6.2. DNA-RNA Heteroduplex

In one aspect, the present disclosure provides a DNA-RNA heteroduplex that can be used for inhibiting expression of androgen receptor mRNA. In some embodiments, the DNA-RNA heteroduplex is used for hair improvement. In some embodiments, the DNA-RNA heteroduplex is used for treatment of a disease associated with expression of androgen receptor (AR).

A DNA-RNA heteroduplex is a double-stranded molecule of a DNA strand and an RNA strand or a modification thereof wherein the DNA strand and the RNA strand or a modification thereof form a double stranded molecule, i.e., a DNA-RNA hybrid. In various embodiments, the DNA strand comprises a sequence specific to an androgen-receptor-specific oligonucleotide (e.g., AR mRNA). For example, the DNA strand can comprise a sequence selected from SEQ ID NO: 1, 3, 5-17. In some embodiments, the DNA strand has a sequence of SEQ ID NO: 1. In some embodiments, the DNA strand has a sequence of SEQ ID NO: 3. The RNA strand can be completely or partially complementary to the DNA strand.

In particular embodiments, the present disclosure provides a DNA-RNA heteroduplex comprising a DNA strand having the sequence of SEQ ID NO: 1 and an RNA strand having the sequence of SEQ ID NO: 2 or a modification thereof. In some embodiments, a DNA-RNA heteroduplex comprises a DNA strand having the sequence of SEQ ID NO: 1 and an RNA strand having the sequence of SEQ ID NO: 2 or a modification thereof.

In some embodiments, the DNA-RNA heteroduplex comprises a DNA strand of SEQ ID NO: 1 and an RNA strand of SEQ ID NO: 2.

In some embodiments, the DNA-RNA heteroduplex comprises a DNA strand of SEQ ID NO: 1 and a modified RNA strand comprising the sequence of SEQ ID NO: 2 with one or more modifications.

In particular embodiments, the present disclosure provides a DNA-RNA heteroduplex comprising a DNA strand having the sequence of SEQ ID NO: 3 and an RNA strand having the sequence of SEQ ID NO: 4 or a modification thereof. In some embodiments, a DNA-RNA heteroduplex comprises a DNA strand having the sequence of SEQ ID NO: 3 and an RNA strand having the sequence of SEQ ID NO: 4 or a modification thereof.

In some embodiments, the DNA-RNA heteroduplex comprises a DNA strand of SEQ ID NO: 3 and an RNA strand of SEQ ID NO: 4.

In some embodiments, the DNA-RNA heteroduplex comprises a DNA strand of SEQ ID NO: 3 and a modified RNA strand comprising the sequence of SEQ ID NO: 4 with one or more modifications.

In some embodiments, the one or more modifications are post-translationally modified. In some embodiments, the one or more modifications are chemical modifications of nucleotides. In some embodiments, the one or more modifications are phosphorothioate substitutions.

In some embodiments, the chemical modification is one or more selected from, without limitation to, the group consisting of the following chemical modifications: modification in which an OH group at the 2′ carbon position of a sugar structure in one or more nucleotides is substituted with any one selected from the group consisting of —CH3 (methyl), OCH3 (methoxy), amine (—NH2), fluorine (—F), —O-2-methoxyethyl, —O-propyl, —O-2-methylthioethyl, —O-3-aminopropyl, —O-3-dimethylaminopropyl, —O—N-methylacetamido and —O-dimethylamidooxyethyl; modification in which oxygen in a sugar structure in nucleotides is substituted with sulfur; modification of a bond between nucleotides to any one bond selected from the group consisting of a phosphorothioate bond, a boranophosphophate bond and a methyl phosphonate bond; modification to PNA (peptide nucleic acid), LNA (locked nucleic acid) or UNA (unlocked nucleic acid); and modification to a DNA-RNA hybrid (Ann. Rev. Med. 55, 61-65 2004; U.S. Pat. Nos. 5,660,985; 5,958,691; 6,531,584; 5,808,023; 6,326,358; 6,175,001; Bioorg. Med. Chem. Lett. 14:1139-1143, 2003; RNA, 9:1034-1048, 2003; Nucleic Acid Res. 31:589-595, 2003; Nucleic Acids Research, 38 (17) 5761-773, 2010; Nucleic Acids Research, 39 (5): 1823-1832, 2011).

In some embodiments, the modified RNA strand comprises one or more phosphorothioated nucleotides. In the case, a phosphorothioate (PS) bond substitutes a sulfur atom for a non-bridging oxygen in the phosphate backbone of the RNA strand. In some embodiments, the modified RNA strand comprises one, two, three, four, five, six, seven, eight, nine or more phosphorothioated nucleotides.

In some embodiments, the modified RNA strand comprises one, two, three, or more phosphorothioated nucleotides in the sequence of CAAACUCU at 3′-end of SEQ ID NO: 2. In some embodiments, the modified RNA strand comprises one, two, three, or more phosphorothioated nucleotides in the sequence of AAACUCU at 3′-end of SEQ ID NO: 2. In some embodiments, the modified RNA strand comprises one, two, three, or more phosphorothioated nucleotides in the sequence of AACUCU at 3′-end of SEQ ID NO: 2. In some embodiments, the modified RNA strand comprises one, two, three, or more phosphorothioated nucleotides in the sequence of ACUCU at 3′-end of SEQ ID NO: 2. In some embodiments, the modified RNA strand comprises one, two, three, or more phosphorothioated nucleotides in the sequence of CUCU at 3′-end of SEQ ID NO: 2. In some embodiments, the modified RNA strand comprises one, two, or three phosphorothioated nucleotides in the sequence of UCU at 3′-end of SEQ ID NO: 2. In some embodiments, each nucleotide of UCU at 3′-end of SEQ ID NO: 2 is a phosphorothioated nucleotide.

In some embodiments, the DNA-RNA heteroduplex further comprises a hydrophobic compound covalently attached to the DNA strand. In some embodiments, the hydrophobic compound is attached to 5′-end of the DNA strand. In some embodiments, the hydrophobic compound is attached to 3′-end of the DNA strand.

In some embodiments, the hydrophobic compound has a molecular weight of 250 to 1,000, and may be any one selected from the group consisting of a steroid derivative, a glyceride derivative, glycerol ether, polypropylene glycol, a C12-C50 unsaturated or saturated hydrocarbon, diacylphosphatidylcholine, a fatty acid, a phospholipid, lipopolyamine, a lipid, tocopherol, and tocotrienol, but is not limited thereto. The steroid derivative may be selected from the group consisting of cholesterol, cholestanol, cholic acid, cholesteryl formate, cholestanyl formate, and cholesteryl amine, and the glyceride derivative may be selected from among mono-, di-, and tri-glycerides and the like. In some embodiments, the fatty acid of the glyceride is a C12-C50 unsaturated or saturated fatty acid. In some embodiments, a C24 hydrocarbon, particularly a hydrophobic hydrocarbon containing a disulfide bond, is used.

In some embodiments, the DNA-RNA heteroduplex further comprises stearyldisulfidehexyl (C₆—S—S—C₁₈) covalently attached to 5′-end of the DNA strand. In some embodiments, the DNA-RNA heteroduplex further comprises stearyldisulfidehexyl (C₆—S—S—C₁₈) covalently attached to 3′-end of the DNA strand.

In some embodiments, the DNA-RNA heteroduplex further comprises a hydrophilic compound covalently attached to the DNA strand. In some embodiments, the hydrophilic compound is attached to 5′-end of the DNA strand. In some embodiments, the hydrophilic compound is attached to 3′-end of the DNA strand.

In some embodiments, the hydrophilic compound is polyethylene glycol (PEG), hexaethylene glycol (HEG), polyvinyl pyrrolidone or polyoxazoline, but the disclosure is not limited thereto.

In some embodiments, the DNA-RNA heteroduplex further comprises hexaethyleneglycol (HEG) covalently attached to 3′-end of the DNA strand. In some embodiments, the DNA-RNA heteroduplex further comprises hexaethyleneglycol (HEG) covalently attached to 5′-end of the DNA strand. In some embodiments, the DNA-RNA heteroduplex further comprises hexaethyleneglycol-(—PO₃-hexaethyleneglycol) covalently attached to 3′-end of the DNA strand. In some embodiments, the DNA-RNA heteroduplex further comprises hexaethyleneglycol-(—PO₃-hexaethyleneglycol) covalently attached to 5′-end of the DNA strand. In some embodiments, the DNA-RNA heteroduplex further comprises hexaethyleneglycol-(—PO₃-hexaethyleneglycol) 2 covalently attached to 3′-end of the DNA strand. In some embodiments, the DNA-RNA heteroduplex further comprises hexaethyleneglycol-(—PO₃-hexaethyleneglycol) 2 covalently attached to 5′-end of the DNA strand. In some embodiments, the DNA-RNA heteroduplex further comprises hexaethyleneglycol-(—PO₃-hexaethyleneglycol) 3 covalently attached to 3′-end of the DNA strand. In some embodiments, the DNA-RNA heteroduplex further comprises hexaethyleneglycol-(—PO₃-hexaethyleneglycol) 3 covalently attached to 5′-end of the DNA strand.

In some embodiments, the hydrophobic compound and the hydrophilic compound are attached to the RNA or DNA strand without a linker. In some embodiments, the hydrophobic compound and the hydrophilic compound are attached to the RNA or DNA strand with a linker, forming a linker-mediated covalent bond.

In some embodiments, the bond between the hydrophobic compound or the hydrophilic compound and the RNA or DNA strand is either a non-degradable bond or a degradable bond. In some embodiments, the non-degradable bond is an amide bond or a phosphate bond. In some embodiments, the degradable bond is any one selected from the group consisting of a disulfide bond, an acid-degradable bond, an ester bond, an anhydride bond, a biodegradable bond, and an enzyme-degradable bond.

The method of generating the method of generating the DNA-RNA heteroduplex is described in PCT/KR2019/015723 and generally in U.S. application Ser. No. 17/057,852, incorporated by reference in their entireties herein.

In some embodiments, the DNA-RNA heteroduplex is SAMiRNA-AR68 or its variant. In some embodiments, the DNA-RNA heteroduplex is SAMiRNA-SRN-021.

In one aspect, the present disclosure provides a DNA strand having the sequence of SEQ ID NO: 1. In some embodiments, the DNA strand can be used without forming a DNA-RNA hybrid to target AR mRNA. In some embodiments, the DNA strand is used to inhibit expression of the AR mRNA. In some embodiments, 5′-end of the DNA strand is covalently attached to stearyldisulfidehexyl (C₆—S—S—C₁₈). In some embodiments, 3′-end of the DNA strand is covalently attached to hexaethylene glycol (HEG) or polyethylene glycol (PEG).

In one aspect, the present disclosure provides an RNA strand having the sequence of SEQ ID NO: 2. In some embodiments, the RNA strand can be used without forming a DNA-RNA hybrid to target AR mRNA. In some embodiments, the RNA strand is used to inhibit expression of the AR mRNA.

In some embodiments, the composition, kit or method of use disclosed herein uses the DNA strand or the RNA strand instead of a DNA-RNA heteroduplex.

### 6.3. Composition of DNA-RNA Heteroduplex

In another aspect, the present disclosure provides a composition comprising the DNA-RNA heteroduplex disclosed herein.

In some embodiments, 0.1-2% by weight of the composition is the DNA-RNA heteroduplex. In some embodiments, 0.25-1.5% by weight of the composition is the DNA-RNA heteroduplex. In some embodiments, 0.25-1.25% by weight of the composition is the DNA-RNA heteroduplex. In some embodiments, 0.25-1% by weight of the composition is the DNA-RNA heteroduplex. In some embodiments, 0.4-0.6% by weight of the composition is the DNA-RNA heteroduplex. In some embodiments, 0.4%, 0.45%, 0.5%, 0.55%, or 0.6% by weight of the composition is the DNA-RNA heteroduplex. In some embodiments, 1%, 1.25%, 1.5%, 1.75% or 2% by weight of the composition is the DNA-RNA heteroduplex.

In some embodiments, 1-20 mg/ml of the composition is the DNA-RNA heteroduplex. In some embodiments, 2.5-10 mg/ml of the composition is the DNA-RNA heteroduplex. In some embodiments, 4-6 mg/ml of the composition is the DNA-RNA heteroduplex. In some embodiments, 4 mg/ml, 4.5 mg/ml, 5 mg/ml, 5.5 mg/ml, or 6 mg/ml of the composition is the DNA-RNA heteroduplex. In some embodiments, 10 mg/ml, 12.5 mg/ml, 1.5 mg/ml, 1.75 mg/ml, or 2 mg/ml of the composition is the DNA-RNA heteroduplex.

In some embodiments, the composition further comprises humectants, hair conditioning or soothing ingredients, and/or one or more buffering ingredients. In some embodiments, the composition comprises one or more humectants selected from betaine and butylene glycol. In some embodiments, the composition comprises one or more hair conditioning or soothing ingredients selected from panthenol, aminopropanol, biotin, niacinamide, and menthol. In some embodiments, the composition comprises one or more buffering ingredients selected from citric acid and sodium citrate.

In some embodiments, the composition comprises denatured ethanol, D-panthenol, betaine, biotin, niacinamide, citric acid, menthol crystals, trisodium citrate dihydrate, and 1,3-butylene glycol. In some embodiments, the composition comprises 30-35% of phosphate buffered saline, 0.4-0.6% of the DNA-RNA heteroduplex, 45-50% of water, 14-15% of ethanol, 0.19-0.2% of panthenol, 1% of niacinamide, 0.02% of citric acid, 0.05% of menthol, 0.03% of sodium citrate, 3% of butylene glycol and less than 0.01% of aminopropanol. In some embodiments, the composition comprises 30-35% of phosphate buffered saline, 0.4-0.6% of the DNA-RNA heteroduplex, 45-50% of water, 14-15% of ethanol, 0.19-0.2% of panthenol, 1% of niacinamide, 0.02% of citric acid, 0.05% of menthol, 0.03% of sodium citrate, and 3% of butylene glycol. In some embodiments, the composition comprises 32.8% of phosphate buffered saline, 0.5-0.6% of the DNA-RNA heteroduplex, 46.3% of water, 14-15% of ethanol, 0.19-0.2% of panthenol, 1% of niacinamide, 0.02% of citric acid, 0.05% of menthol, 0.03% of sodium citrate, 3% of butylene glycol and less than 0.01% of aminopropanol.

In some embodiments, the composition is in the form of ointment, paste, gel, jelly, serum, aerosol spray, non-aerosol spray, foam, cream, lotion, solution, or suspension formulation.

In some embodiments, the composition has a pH between 5 and 7. In some embodiments, the composition has a pH between 5.5 and 6.5 or between 6 and 6.5. In some embodiments, the composition has a pH of 6. In some embodiments, the composition has a pH of 6.29. In some embodiments, the composition has a pH of 6.50. In some embodiments, the composition has a pH of 7. In some embodiments, the composition has a pH of 7.02.

In some embodiments, the DNA-RNA heteroduplex in the composition forms self-assembled micelle inhibitory RNA (SAMiRNA) nanoparticle-type siRNA through the hydrophobic interaction of the hydrophobic material (e.g., stearyldisulfidehexyl (C₆—S—S—C₁₈)). In some embodiments, the DNA-RNA heteroduplex in the composition does not form nanoparticle-type siRNA. In some embodiments, some, but not all, DNA-RNA heteroduplex in the composition forms SAMiRNA.

In some embodiments, the DNA-RNA heteroduplex forms a nanoparticle. In some embodiments, the nanoparticle has a mean diameter between 50 and 200 nm. In some embodiments, the nanoparticle has a mean diameter between 50 and 150 nm, between 75 and 125 nm, between 80 and 120 nm, between 90 and 110 nm, or between 100 and 110 nm.

In some embodiments, the composition is directly applied to a subject. In some embodiments, the composition is diluted before application to a subject.

### 6.4. Kit

In one aspect, the present disclosure provides a kit for hair improvement. In some embodiments, the kit comprises a container and the composition of the DNA-RNA heteroduplex disclosed herein. In some embodiments, the container contains a unit dose of the composition of the DNA-RNA heteroduplex. In some embodiments, the container contains multiple unit doses of the composition of the DNA-RNA heteroduplex.

In some embodiments, the container comprises a dispenser for delivering the composition. In some embodiments, the container comprises a pump dispenser for delivering the composition. In some embodiments, the container comprises a pump dispenser for delivering the composition. In some embodiments, the container comprises a piston for delivering a unit dose of the composition. In some embodiments, the container is configured to deliver a unit dose of the composition at a time.

In some embodiments, the container comprises a tool for massage. In some embodiments, the massage tool is made of silicon. In some embodiments, the tool is configured to massage scalp.

### 6.5. Method of Use

In one aspect, the present disclosure provides a method of using the DNA-RNA-heteroduplex or its composition. It can be used to inhibit expression of AR mRNA. In some embodiments, the method comprises the step of applying the DNA-RNA-heteroduplex or its composition to inhibit expression of AR mRNA locally or systematically. In some embodiments, the DNA-RNA-heteroduplex or its composition is applied locally on the skin. In some embodiments, the DNA-RNA-heteroduplex or its composition is applied topically.

In some embodiments, the DNA-RNA-heteroduplex or its composition is applied to a subject having a symptom associated with expression of AR mRNA. In some embodiments, the DNA-RNA-heteroduplex or its composition is applied to a subject to treat a disease associated with expression of AR mRNA. In some embodiments, the subject is a human or an animal.

In some embodiments, present disclosure provides a method of hair improvement, comprising applying the composition disclosed herein on scalp of a subject. In some embodiments, the scalp is massaged after application of the composition. In some embodiments, the scalp is massaged for one, two, three, four, five, six, seven, eight, nine, or ten minutes.

The composition can be applied once a week, once per two weeks or once per four weeks. In some embodiments, the composition is applied once a month, once in two months, once in three months, once in four months, once in five months, once in six months, once in seven months, once in eight months, once in nine months, once in ten months, once in eleven months, or once in twelve months.

In some embodiments, the composition is applied once a week for at least four months. In some embodiments, the composition is applied once a week for at least five months. In some embodiments, the composition is applied once a week for at least six months. In some embodiments, the composition is applied once a week for at least seven months. In some embodiments, the composition is applied once a week for at least eight months. In some embodiments, the composition is applied once a week for at least nine months. In some embodiments, the composition is applied once a week for at least ten months.

In some embodiments, the composition is applied for two months, three months, four months, five months, six months, seven months, eight months, nine months, ten months, eleven months, or twelve months. In some embodiments, the composition is applied for more than one year.

In some embodiments, the composition is applied once a week for four months. In some embodiments, the composition is applied once a week for five months. In some embodiments, the composition is applied once a week for six months. In some embodiments, the composition is applied once a week for seven months.

In some embodiments, the composition is used less frequent after the maximum effect of the composition is achieved.

In some embodiments, the composition is applied once a week for four months, and after the four months less than once a week. In some embodiments, the composition is applied once a week for four months, and after the four months once per 2 weeks, once per 3 weeks or once per 4 weeks.

In some embodiments, the composition is applied once a week for four months, and after the four months less than once a week. In some embodiments, the composition is applied once a week for four months, and after the four months once per 1 month, once per 2 months, once per 3 months, once per 4 months, once per 5 months, once per 6 months, or less.

In some embodiments, the composition is applied once a week for four months, and after the four months discontinued.

In some embodiments, the subject has alopecia. In some embodiments, the subject has moderate androgenetic alopecia. In some embodiments, the subject has Norwood scale stadium at least III or more increased hair loss. In some embodiments, the subject has Ludwig scale stadium at least I-3 or more increased hair loss.

In some embodiments, the subject is a man. In some embodiments, the subject is a woman.

In some embodiments, the composition applied to the subject comprises the DNA-RNA heteroduplex at 1-50 mg/ml. In some embodiments, the composition applied to the subject comprises the DNA-RNA heteroduplex at 1-40 mg/ml. In some embodiments, the composition applied to the subject comprises the DNA-RNA heteroduplex at 1-30 mg/ml. In some embodiments, the composition applied to the subject comprises the DNA-RNA heteroduplex at 1-20 mg/ml. In some embodiments, the composition applied to the subject comprises the DNA-RNA heteroduplex at 2.5-10 mg/ml. In some embodiments, the composition applied to the subject comprises the DNA-RNA heteroduplex at 4 mg/ml, 4.5 mg/ml, 5 mg/ml, 5.5 mg/ml, or 6 mg/ml. In some embodiments, the composition applied to the subject comprises the DNA-RNA heteroduplex at 4.5 mg/ml, 5 mg/ml, or 5.5 mg/ml.

In some embodiments, the method comprises applying 0.1-10 ml of the DNA-RNA heteroduplex more than once a week. In some embodiments, the method comprises applying 0.1-7.5 ml of the DNA-RNA heteroduplex more than once a week. In some embodiments, the method comprises applying 0.5-5 ml of the DNA-RNA heteroduplex more than once a week. In some embodiments, the method comprises applying 0.5-3 ml of the DNA-RNA heteroduplex more than once a week. In some embodiments, the method comprises applying 0.5-2 ml of the DNA-RNA heteroduplex more than once a week. In some embodiments, the method comprises applying 0.5-1.5 ml of the DNA-RNA heteroduplex more than once a week. In some embodiments, the method comprises applying 1-1.5 ml of the DNA-RNA heteroduplex more than once a week.

In some embodiments, the method comprises applying 1-100 mg of the DNA-RNA heteroduplex more than once a week. In some embodiments, the method comprises applying 1-100 mg of the DNA-RNA heteroduplex once a week. In some embodiments, the method comprises applying 1-100 mg of the DNA-RNA heteroduplex less than once a week.

In some embodiments, the method comprises applying 1-100 mg of the DNA-RNA heteroduplex once a week. In some embodiments, the method comprises applying 1-50 mg of the DNA-RNA heteroduplex once a week. In some embodiments, applying 1-40 mg of the DNA-RNA heteroduplex once a week. In some embodiments, the method comprises applying 1-30 mg of the DNA-RNA heteroduplex once a week. In some embodiments, the method comprises applying 1-20 mg of the DNA-RNA heteroduplex once a week. In some embodiments, the method comprises applying 1-15 mg of the DNA-RNA heteroduplex once a week. In some embodiments, the method comprises applying 1-10 mg of the DNA-RNA heteroduplex once a week. In some embodiments, the method comprises applying 1-5 mg of the DNA-RNA heteroduplex once a week. In some embodiments, the method comprises applying 2-10 mg of the DNA-RNA heteroduplex once a week. In some embodiments, the method comprises applying 2.5-7.5 mg of the DNA-RNA heteroduplex once a week.

In some embodiments, the method comprises applying 1-100 mg of the DNA strand or the RNA strand once a week. In some embodiments, the method comprises applying 1-50 mg of the DNA strand or the RNA strand once a week. In some embodiments, the method comprises applying 1-25 mg of the DNA strand or the RNA strand once a week. In some embodiments, the method comprises applying 1-15 mg of the DNA strand or the RNA strand once a week.

In some embodiments, the method comprises applying 1-50 mg of the DNA-RNA heteroduplex per area scalp (cm²) once a week. In some embodiments, the method comprises applying 1-40 mg of the DNA-RNA heteroduplex per area scalp (cm²) once a week.

In some embodiments, the method comprises applying 1-200 mg of the DNA-RNA heteroduplex per area scalp (cm²) once a week. In some embodiments, the method comprises applying 1-150 mg of the DNA-RNA heteroduplex per area scalp (cm²) once a week. In some embodiments, the method comprises applying 1-100 mg of the DNA-RNA heteroduplex per area scalp (cm²) once a week. In some embodiments, the method comprises applying 1-50 mg of the DNA-RNA heteroduplex per area scalp (cm²) once a week. In some embodiments, the method comprises applying 1-30 mg of the DNA-RNA heteroduplex per area scalp (cm²) once a week. In some embodiments, the method comprises applying 5-20 mg of the DNA-RNA heteroduplex per area scalp (cm²) once a week. In some embodiments, the method comprises applying 6-15 mg of the DNA-RNA heteroduplex per area scalp (cm²) once a week. In some embodiments, the method comprises applying 8-10 mg of the DNA-RNA heteroduplex per area scalp (cm²) once a week.

In some embodiments, the method comprises applying 0.5-5 ml of the DNA-RNA heteroduplex per area scalp (cm²) once a week. In some embodiments, the method comprises applying 1-2 ml of the DNA-RNA heteroduplex per area scalp (cm²) once a week. In some embodiments, the method comprises applying 1-1.5 ml of the DNA-RNA heteroduplex per area scalp (cm²) once a week.

### 7. EXAMPLES

### 7.1. Screening of Potent SAMiRNA for AR Silencing

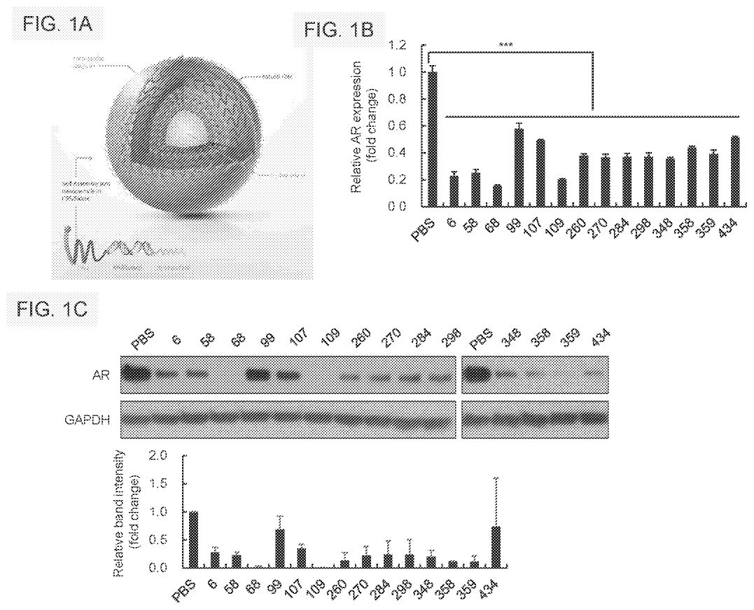

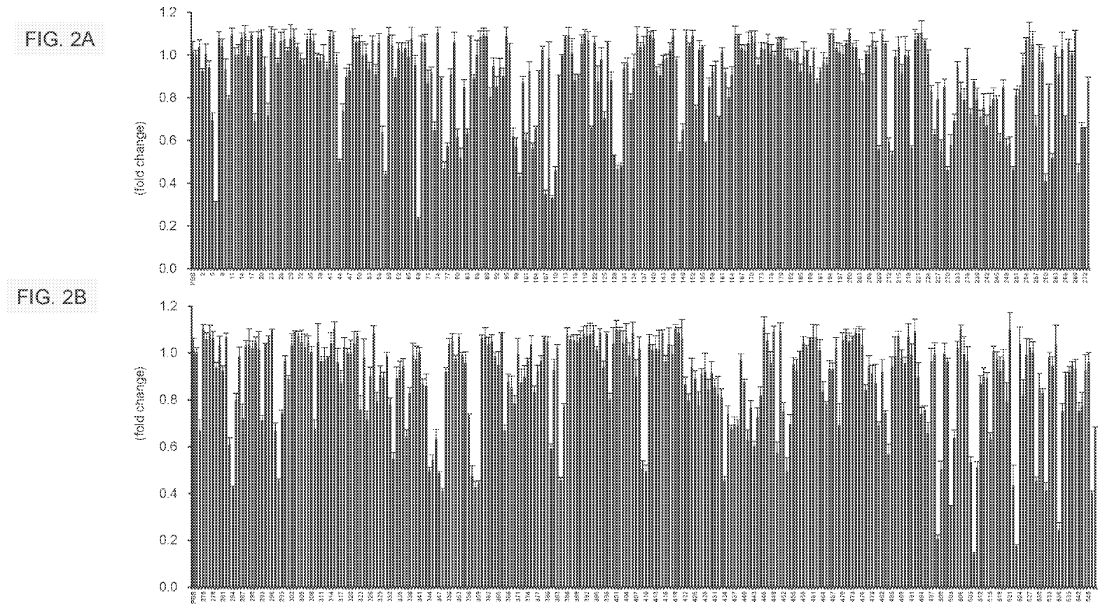

SAMiRNA nanoparticles, as shown in FIG. 1A, consist of hydrophilic polymer polyethylene glycol (PEG) and hydrophobic hydrocarbon conjugates at each end of an unmodified DNA/RNA heteroduplex. For potent SAMiRNAs specifically targeting AR, SAMiRNAs containing 19 base pairs of DNA/RNA heteroduplexes were designed by the sliding window algorithm and selected for specificity for AR mRNA. A total of 547 SAMIRNA candidate-conjugated sense strands and antisense strands were synthesized, purified and annealed to nanoparticles. The silencing effect of AR in LNCap cells was analyzed for each SAMiRNA candidate by reverse transcription-quantitative polymerase chain reaction (RT-qPCR). Fourteen SAMiRNA candidates were selected based on their knockdown efficiency (>50% AR silencing efficacy) (FIG. 1B and FIG. 2). In addition, we verified the decreased level of AR protein in fourteen SAMiRNA candidate-treated LNCap cells (FIG. 1C). AR68 and AR109 were found to be the most potent SAMiRNAs. AR68 has SEQ ID NO: 3 as a sense strand and AR109 has SEQ ID NO: 5 as a sense strand.

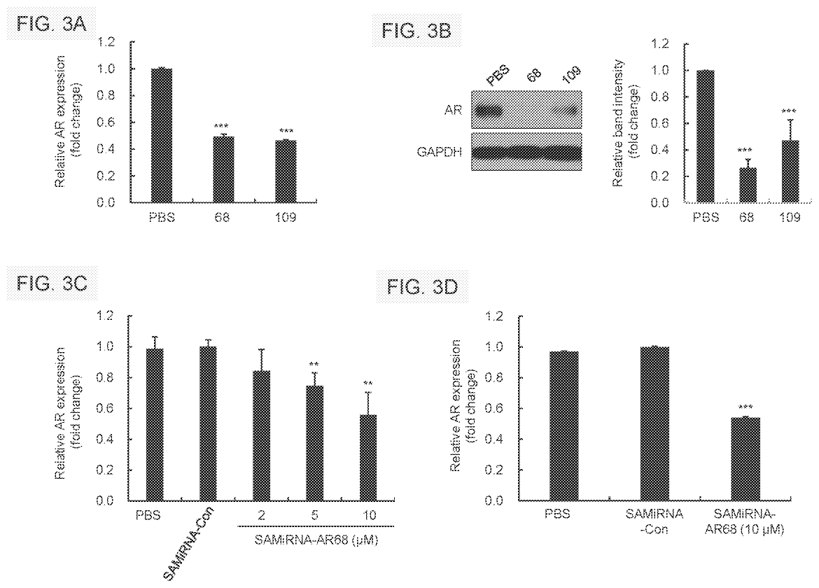

To confirm efficacy in human hair cells, human follicle dermal papilla (HFDP) cells were treated with AR68 and AR109, which significantly decreased AR mRNA and protein levels (FIGS. 3A and 3B). As shown in FIG. 2B, AR68 reduced AR protein levels more effectively than AR109 in HFDP cells, confirming that AR68 is the more potent siRNA sequence for AR silencing. Treatment of HFPD cells with AR68 reduced AR mRNA expression in a dose-dependent manner (FIG. 3C), and the AR protein inhibitory effect was confirmed by enzyme-linked immunosorbent assays (ELISAs) (FIG. 3D).

### 7.2. SAMIRNA-AR68 Decreased the AR mRNA and Protein Levels in Human Hair Follicles

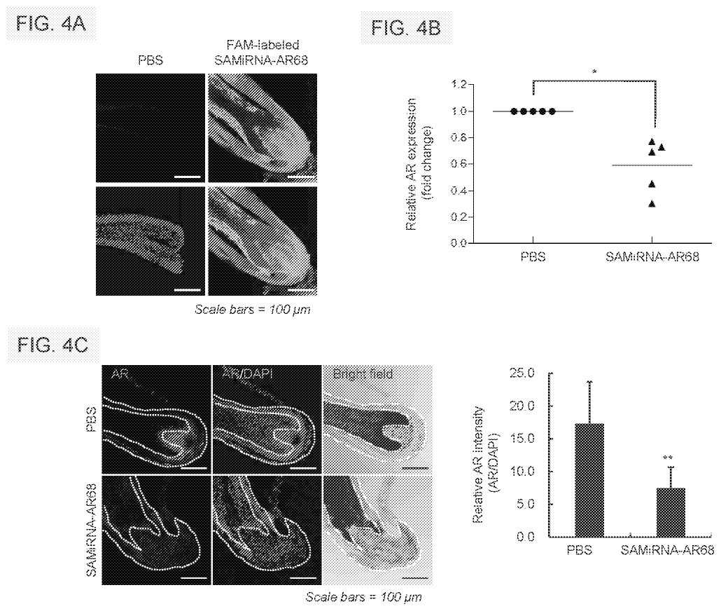

To evaluate SAMiRNA nanoparticle delivery to human hair follicles, fluorescein (FAM)-labeled AR68 was applied to plucked hair and visualized the samples by confocal microscopy. Plucked hair often lacks the dermal papilla compared with microdissected hair follicles. We plucked multiple hairs and selected hairs with a hair bulb. FAM-labeled AR68 was added to plucked hairs with dermal papilla. As expected, FAM-labeled AR68 was efficiently delivered to the outer root sheath (ORS) as well as the dermal papilla of the hair bulb (FIG. 4A). AR silencing in hair follicles was confirmed by RT-qPCR analysis (FIG. 4B). Immunofluorescence analysis of the AR68-treated plucked hair showed a substantial reduction in AR protein expression (FIG. 4C). The AR protein is highly expressed in the dermal papilla, and transactivation of AR by binding to DHT in dermal papilla cells induces apoptosis that promotes AGA progression. Therefore, we confirmed that AR68 nanoparticles at 10 μM could be delivered and silenced AR in dermal papilla cells of hair follicles. The efficacy of AR68 was investigated at higher concentrations for a clinical study of AGA treatment.

### 7.3. The Safety of SAMiRNA-AR68 Nanoparticles

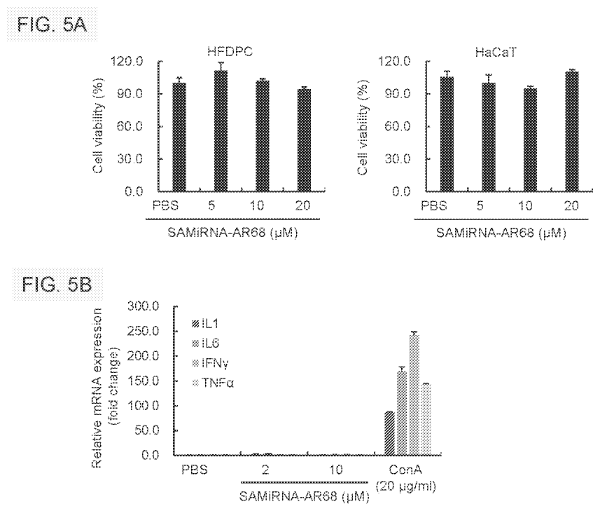

One of the limitations in developing RNAi therapeutics for clinical applications is cytotoxicity and systemic toxicity, including innate immune stimulation. To evaluate the cytotoxicity of AR68, a cell viability assay was performed in HFDP and keratinocyte HaCaT cells at effective concentrations (10 μM). Neither HFDP nor HaCaT cells showed cytotoxicity up to 20 μM AR68 (FIG. 5A). It was investigated whether AR68 induces nonspecific innate immune stimulation in human PBMCs. AR68 was treated at concentrations up to 10 μM, and proinflammatory cytokines related to TLR3 signaling were analyzed after 6 hrs. AR68 did not induce proinflammatory cytokines, including interleukin (IL)-1B, IL-6, interferon-gamma (INF-γ), and tumor necrosis factor-alpha (TNF-α), in PBMCs compared to nonstimulated negative controls; as a positive control, concanavalin A significantly induced proinflammatory cytokines (FIG. 5B). Taken together, these results demonstrate that AR68 does not induce cytotoxicity or innate immune stimulation at 10 μM, and these results guided determination of the effective dose of AR68 to be used in clinical applications.

### 7.4. The Stability of SAMiRNA-AR68 Nanoparticles

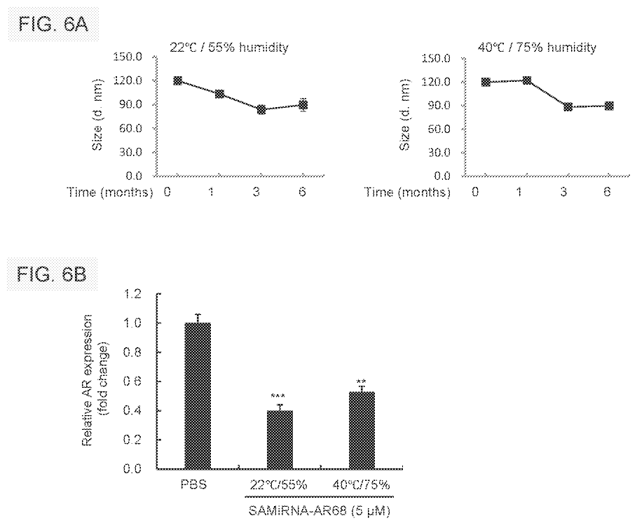

The storage stability of SAMiRNA solution was tested at various temperatures. The nanoparticle size of SAMiRNA was monitored by qNano Gold to evaluate the long-term stability of AR68 nanoparticles. The average size of the AR68 nanoparticles was approximately 99.2±5.1 nm (22° C., 55%±5 humidity) and 105.0±2.5 nm (40° C., 75%±5 humidity). The AR68 formulation remained stable for 6 months (FIG. 6A). Further, AR knockdown was tested by RT-qPCR using AR68 samples stored for 6 months under the indicated conditions. As expected, it showed a similar inhibitory effect of AR mRNA in HFDPCs (FIG. 6B).

### 7.5. Clinical Study I: Low-Dose SAMiRNA-AR68 (0.5 mg/ml) Treatment Three Times Per Week

### General Characteristics of the Subjects

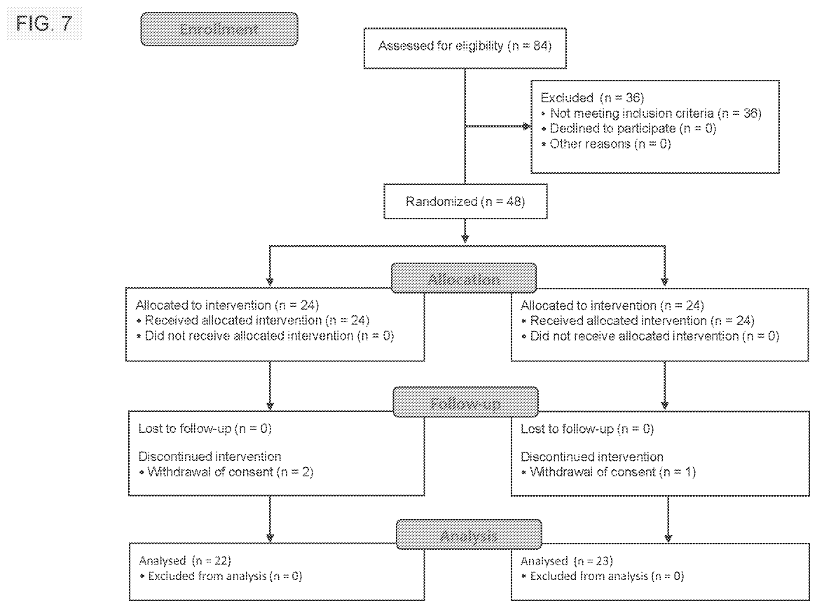

A total of 48 male and female subjects diagnosed with moderate androgenetic alopecia were recruited and randomly assigned to an AR68 low-dose treatment group (test group) (n=24) or placebo group (n=24). In the AR68 low-dose treatment group, 2 of 24 subjects dropped out (withdrawal of consent); thus, 22 subjects completed the study. The mean age was 42.4±8.10 years, and the male to female ratio was 8:14. In the placebo group, one of 24 subjects dropped out (withdrawal of consent), with 23 completing the study. The mean age of the placebo group was 42.4±6.69 years, and the male to female ratio was 6:17. There was no statistically significant difference between the two groups based on group homogeneity analysis performed to confirm the validity of randomization (FIG. 7 and Table 1).

TABLE 1
Demographic characteristics of the subjects in clinical study I.

| | | AR68 0.5 mg/ml | | Placebo | | |
|---|---|---|---|---|---|---|
| | | (n = 22) | | (n = 23) | | p- |
| Characteristics | | n (%) | | n (%) | | Value |
| Sex | Male | 8 | (36.36) | 6 | (26.09) | 0.530 |
| | Female | 14 | (63.64) | 17 | (73.91) | |
| Age | Mean ± SD | 42.4 ± 8.10 | | 42.4 ± 6.69 | | 0.546 |
| | Median | 44.00 | | 43.00 | | |
| | Min, Max | 22.00, 53.00 | | 26.00, 53.00 | | |
| | 20< | 0 | (0.00) | 0 | (0.00) | |
| | 20-29 | 2 | (9.09) | 1 | (4.35) | |
| | 30-39 | 3 | (13.64) | 6 | (26.09) | |
| | 40-49 | 14 | (63.63) | 13 | (56.52) | |
| | ≥50 | 3 | (13.64) | 3 | (13.04) | |

### Analysis of Hair Density and Total Hair Counts

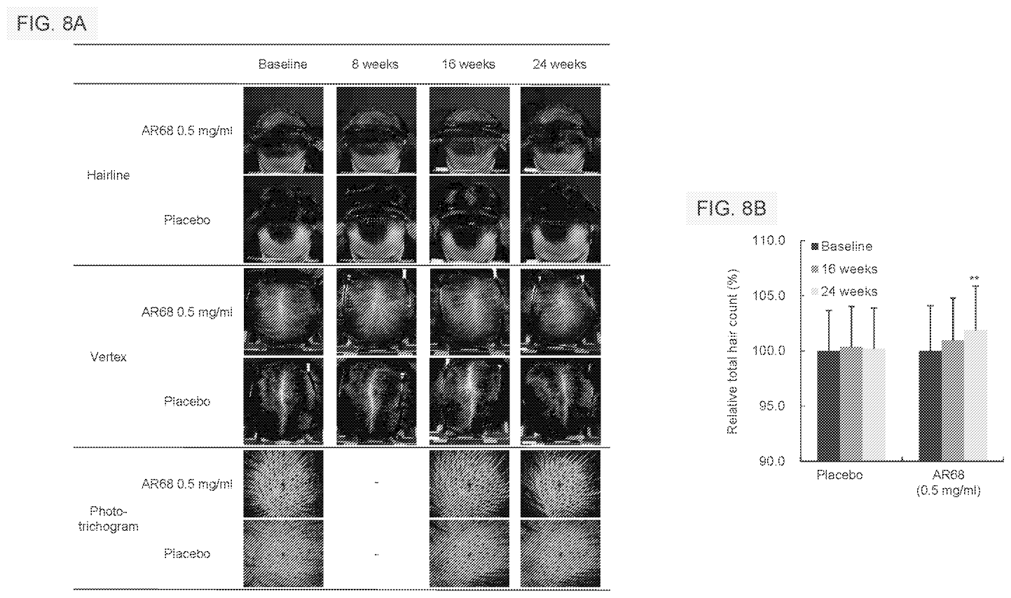

Photographic assessment revealed increased hair density in the AR68 low-dose treatment group compared with that at baseline (FIG. 8A and Table 2a). As shown by phototrichogram analysis, the total hair count increased at 24 weeks after treatment with AR68 compared to that at baseline (from 133.14 to 135.41 hairs/cm²; p<0.01) (Table 2b). The mean change and rate of total hair count increased by 2.273±3.089 and 1.870% at 24 weeks in the AR68 0.5 mg/ml treatment group, respectively, compared to those at baseline (FIG. 8B and Table 3).

TABLE 2
Descriptive statistical analysis of hair density (a) and total
hair counts (b) between time points in clinical study I.

| | | Base- | 8 | 16 | 24 |
|---|---|---|---|---|---|
| | | line | weeks | weeks | weeks |
| (a) | | | | | |
| AR68 0.5 | Mean¹ | 0.000 | 0.000 | 0.045 | 0.045 |
| mg/ml | SD | 0.000 | 0.000 | 0.213 | 0.375 |
| (n = 22) | p-value | — | 0.317 | 0.083 | 0.317 |
| Placebo | Mean | 0.000 | 0.000 | 0.043 | 0.087 |
| (n = 23) | SD | 0.000 | 0.000 | 0.209 | 0.417 |
| | p-value | — | 1.000 | 1.000 | 0.317 |
| (b) | | | | | |
| AR68 0.5 | Mean(n/cm²)² | 133.14 | — | 134.00 | 135.41 |
| mg/ml | SD | 25.64 | — | 24.21 | 25.48 |
| (n = 22) | p-value³ | — | — | 0.078 | 0.002** |
| Placebo | Mean(n/cm²) | 129.26 | — | 129.70 | 129.57 |
| (n = 23) | SD | 22.64 | — | 22.66 | 22.96 |
| | p-value | — | — | 0.441 | 0.588 |

¹Increment of mean value represents improvement of hair condition on hairline and vertex
²Increment of mean value represents improvement of hair counts
³Significantly different at **p < 0.01 compared with baseline

TABLE 3
Statistical analysis of Δ total hair counts (weeks-Baseline)
between SAMiRNA-AR68 0.5 mg/ml treated and placebo groups.

| | AR68 0.5 mg/ml | Placebo | |
|---|---|---|---|
| Δ Total hair counts | (n = 22) | (n = 23) | |
| (n/cm²) | Mean ± SD | Mean ± SD | p-value¹ |
| 16 weeks | 0.864 ± 2.189 | 0.435 ± 2.660 | 0.477 |
| 24 weeks | 2.273 ± 3.089 | 0.304 ± 2.653 | 0.043* |

¹Significantly different at *p < 0.05 compared with AR 0.5 mg/ml treated group and placebo group at same time point

### Subject Self-Assessment Questionnaires

There was no significant difference between the AR68 0.5 mg/ml treatment group and the placebo group.

### Safety Assessments

In the safety assessment, no adverse events in any of the 45 subjects were observed during the clinical study period.

### 7.6. Clinical Study II: High-Dose SAMiRNA-AR68 (5 mg/ml) Treatment Once a Week

### General Characteristics of the Subjects

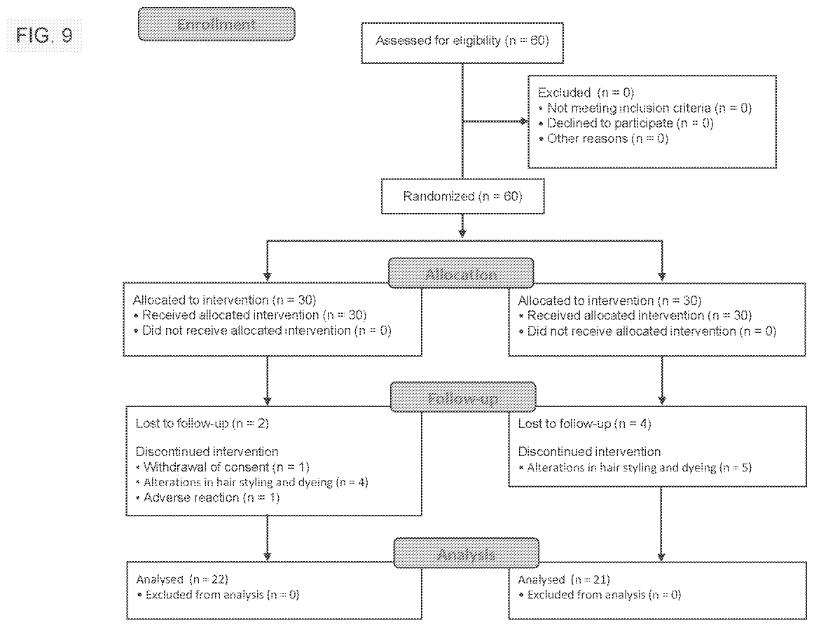

To increase efficacy and user convenience, we designed and conducted a clinical study with high-dose (5 mg/ml) treatment once per week. A total of 60 male and female subjects diagnosed with moderate androgenetic alopecia participated and were randomly assigned to an AR68 5 mg/ml treatment group (n=30) or a placebo group (n=30). During the study period, six subjects withdrew (withdrawal of consent or loss to follow-up), ten subjects withdrew at the investigator's discretion (hair perm, dyeing, etc.), and one subject dropped out due to an adverse reaction; thus, 43 subjects completed the clinical study. In the AR68 treatment group, 8 of 30 subjects dropped out, and 22 completed the study. The average age was 44.77±10.88 years, and the male to female ratio was 9:13. In the placebo group, 9 of 30 subjects dropped out, and 21 completed the study; the mean age was 46.48±7.93 years, and the male to female ratio was 10:11. There was no statistically significant difference between the two groups, validating the randomization (FIG. 9 and Table 4).

TABLE 4
Demographic characteristics of the subjects in clinical study II.

| | | AR68 5 mg/ml (n = 22) | | Placebo (n = 21) | |
|---|---|---|---|---|---|
| Characteristics | | n (%) | | n (%) | |
| Sex | Male | 9 | (40.90) | 10 | (47.62) |
| | Female | 13 | (50.10) | 11 | (52.38) |
| Age | Mean ± SD | 44.77 ± 10.88 | | 46.48 ± 7.93 | |
| | Median | 49.50 | | 48.00 | |
| | Min, Max | 22.00, 54.00 | | 25.00, 54.00 | |
| | 20< | 0 | (0.00) | 0 | (0.00) |
| | 20-29 | 4 | (18.18) | 2 | (9.52) |
| | 30-39 | 1 | (4.55) | — | |
| | 40-49 | 6 | (27.27) | 9 | (42.86) |
| | ≥50 | 11 | (50.00) | 10 | (47.62) |

### Analysis of Total Hair Counts and Photographic Assessments

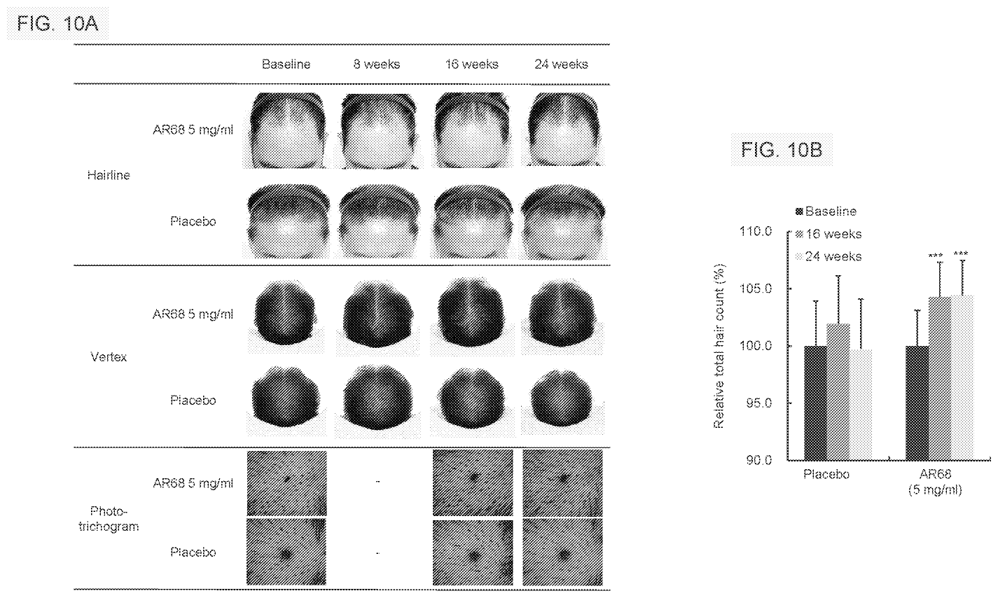

Hair density by photo assessment was significantly increased at eight weeks (0.091±0.294), 16 weeks (0.159±0.29; p<0.05), and 24 weeks in the AR68 5 mg/ml treatment group compared to that at baseline (before the assessment). There was a significant improvement at week 24 (0.250±0.551; p<0.05) (FIG. 10A and Table 5a). Phototrichogram analysis showed significantly higher total hair counts for the AR68 treatment group at 16 weeks (from 182.182 to 189.727 hairs/cm²; p<0.001) and 24 weeks (from 182.182 to 189.909 hairs/cm²; p<0.001) (Table 4b) than that at baseline. The mean change and rate of total hair count increased by 7.545±7.896 and 4.264% (p<0.001) at 16 weeks and 7.727±8.659 and 4.421% (p<0.001) at 24 weeks in the AR68 5 mg/ml treatment group, respectively, compared to those at baseline (FIG. 10B and Table 6).

TABLE 5
Descriptive statistical analysis of hair density (a) and total
hair counts (b) between time points in clinical study II.

| | | Base- | 8 | 16 | 24 |
|---|---|---|---|---|---|
| | | line | weeks | weeks | weeks |
| (a) | | | | | |
| AR68 5 | Mean¹ | 0.000 | 0.091 | 0.159 | 0.250 |
| mg/ml | SD | 0.000 | 0.294 | 0.358 | 0.551 |
| (n = 22) | | | | | |
| Placebo | Mean | 0.000 | −0.095 | −0.071 | −0.071 |
| (n = 21) | SD | 0.000 | 0.340 | 0.396 | 0.396 |
| | p-value³ | — | 0.098 | 0.039* | 0.035* |
| (b) | | | | | |
| AR68 5 | Mean(n/cm²)² | 182.182 | — | 189.727 | 189.909 |
| mg/ml | SD | 26.626 | — | 27.216 | 27.154 |
| (n = 22) | p-value⁴ | — | — | <0.001*** | <0.001*** |
| Placebo | Mean(n/cm²) | 181.714 | — | 185.429 | 181.524 |
| (n = 21) | SD | 32.661 | — | 35.778 | 36.371 |
| | p-value | — | — | — | — |

¹Increment of mean value represents improvement of hair condition on hairline and vertex
²Increment of mean value represents improvement of hair counts
³Significantly different at *p < 0.05 compared with AR68 5 mg/ml treated group and placebo group at same time point
⁴Significantly different at ***p < 0.001 compared with baseline

TABLE 6
Statistical analysis of Δ total hair counts (weeks-Baseline)
between SAMiRNA-AR68 5 mg/ml treated and placebo groups.

| | AR68 5 mg/ml | Placebo | |
|---|---|---|---|
| Δ Total hair counts | (n = 22) | (n = 21) | |
| (n/cm²) | Mean ± SD | Mean ± SD | p-value¹ |
| 16 weeks | 7.545 ± 7.896 | 3.714 ± 10.335 | 0.279 |
| 24 weeks | 7.727 ± 8.659 | −0.190 ± 12.875 | 0.026* |

¹Significantly different at *p < 0.05 compared with AR68 5 mg/ml treated group and placebo group at same time point

Subject self-assessment questionnaires

Regarding the efficacy of product use, the subjects in the AR68 5 mg/ml treatment group gave positive feedback in the subject self-assessment questionnaires. The participants responded that they were “satisfied with the sample used” at 8 (72.73%), 16 (81.82%), and 24 (59.09%) weeks. At weeks 8 (36.36%), 16 (63.64%) and 24 (50.00%), “the feeling of fuller hair in the vertex area” was reported, and hair loss decreased at weeks 8 (45.45%), 16 (54.55%) and 24 (72.73%).

### Safety Assessments

In the safety assessment, one subject in the AR68 5 mg/ml treatment group developed erythema, edema, and itching at the test site, but the symptoms were relieved after diagnosis and treatment by a dermatologist. The dermatologist's diagnosis determined that the relationship between the symptoms and the test substance was slight.

### 7.7. Methods

### Cell Culture and Reagents

Human prostate cancer LNCaP and human keratinocyte HaCaT cells were purchased from the American Type Culture Collection (ATCC), and human follicle dermal papilla cells (HFDPCs) were purchased from PromoCell (C-12071). Human peripheral blood mononuclear cells (PBMCs) were obtained from Cellular Technology Limited (CTL-UP1). LNCaP cells were cultured in RPMI medium (HyClone) supplemented with 1% penicillin-streptomycin (HyClone) and 10% fetal bovine serum (FBS, HyClone). HaCaT cells were cultured in DMEM (HyClone) supplemented with 1% penicillin-streptomycin and 10% FBS. HFDPCs were maintained in follicle dermal papilla cell growth medium (PromoCell). Individual human hairs were pulled out with forceps in the occipital area of the scalp, and plucked hairs with visible bulbs were selected by microscopy. The plucked hair follicles were cultured in DMEM/F12 (Gibco) supplemented with 1% penicillin-streptomycin, 10% FBS, 10 μg/mL insulin-transferrin-selenium-X supplement (Gibco), 2.5 μg/mL amphotericin B (Gibco), 1% GlutaMAX (Gibco), 20 ng/mL fibroblast growth factor (FGF, PeproTech), 20 ng/ml epidermal growth factor (EGF, Sigma), and 10 ng/mL hydrocortisol (Tokyo Chemical Industry). Plucked human hair follicles were treated with 10 μM SAMiRNA-AR68 for 48 hrs followed by qPCR and immunofluorescence analysis. The synthesis and quality control of the SAMIRNA nanoparticles were previously described.

### Measurement of SAMiRNA Nanoparticle Size

For determination of the long-term stability of SAMiRNA, the nanoparticle size was monitored by qNano Gold (Izon Science) according to standard operating procedures. Briefly, 35 μl of SAMiRNA-AR68 was analyzed with qNano Gold equipment using an NP80 Nanopore (Izon Science) and applying a 47 mm stretch, a current of 140 nA, and 10 mBar parametric conditions. The calibration particles (CPC100, Izon Science) were assayed before the experimental samples under identical conditions. Particle counts (250 events each) were finally determined using the qNano software provided by Izon Science (Izon Control Suite Version 3.3).

### Reverse Transcription and Quantitative Polymerase Chain Reaction (RT-qPCR)

RT-qPCR was performed according to the MIQE guidelines. Total RNA from cells was extracted using an AccuPrepR Universal RNA Extraction Kit (K-3140, Bioneer) according to the manufacturer's instructions. For total RNA extraction from hair follicles, SAMIRNA-AR68-treated plucked hair follicles were washed with PBS, resuspended in TRIzol® reagent (Invitrogen), homogenized with a Biomasher II® Disposable Micro Tissue Homogenizer (Polyscience), and purified with an AccuPrep® Universal RNA Extraction Kit (K-3140, Bioneer) according to the manufacturer's instructions. Total RNA (1 μg) was reverse transcribed using Accupower® RocketScript™ Cycle RT PreMix (dT20) in a 20 μl reaction (K-2201, Bioneer) according to the manufacturer's instructions. For qPCR analysis, 10 μl of 10-fold diluted cDNA was amplified using AccuPower® 2× GreenStar™ qPCR MasterMix (K-6253, Bioneer). The following primer sets were used: AR, forward 5′-TTGTACACGTGGTCAAGTGG-3′ (SEQ ID NO: 18) and reverse 5′-TGGAGTTGACATTGGTGAAGG-3′ (SEQ ID NO: 19); RPLPO, forward 5′-TGCCATTGCCCCATGTGAAG-3′ (SEQ ID NO: 20) and reverse 5′-AGCTGCACATCACTCAGGATT-3′ (SEQ ID NO: 21); IL-1B, forward 5′-CTGAGCTCGCCAGTGAAAT-3′ (SEQ ID NO: 22) and reverse 5′-CTGTAGTGGTGGTCGGAGA-3′ (SEQ ID NO: 23); IL-6, forward 5′-AGATGCAATAACCACCCCTG-3′ (SEQ ID NO: 24) and reverse 5′-TGCGCAGAATGAGATGAGTT-3′ (SEQ ID NO: 25); TNF, forward 5′-CTGTAGCCCATGTTGTAGCA-3′ (SEQ ID NO: 26) and reverse 5′-GGTTATCTCTCAGCTCCACG-3′ (SEQ ID NO: 27); IFNG, forward 5′-GAATGTCCAACGCAAAGCAA-3′ (SEQ ID NO: 28) and reverse 5′-ACCTCGAAACAGCATCTGAC-3′ (SEQ ID NO: 29); RPL13A, forward 5′-TGCCATTGCCCCATGTGAAG-3′ (SEQ ID NO: 20) and reverse 5′-AGCTGCACATCACTCAGGATT-3′ (SEQ ID NO: 21); IL-1B, forward 5′-GTGTTTGACGGCATCCCACC-3′ (SEQ ID NO: 30) and reverse 5′-TAGGCTTCAGACGCACGACC-3′ (SEQ ID NO: 31). PCR amplification was performed in a 50 μl reaction as follows: one cycle at 95° C. for 10 min, followed by 40 cycles at 95° C. for 5 s, 58° C. for 25 s, and 72° C. for 30 s, with one final extension step at 72° C. for 5 min. All experiments were performed in triplicate. The delta-delta Ct method was used to determine relative fold changes, and all data were normalized to the internal control gene.

### Immunoblot Analysis

LNCaP and HFDP cells were collected and lysed using cell lysis buffer (Cell Signaling Technology) with protease inhibitor cocktail (Thermo Fisher Scientific). Proteins were separated by 10% sodium dodecyl sulfate-polyacrylamide gel electrophoresis (SDS-PAGE) and transferred to a PVDF membrane (Bio-Rad). After the membranes were blocked with 5% skim milk in Tris-buffered saline (TBS) for at least 1 hr, they were incubated with the indicated antibodies overnight, washed with TBS three times, and then incubated with horseradish peroxidase-conjugated secondary antibodies for 2 hrs. Immunoblotting was performed with the following antibodies: anti-AR (ab133273, Abcam), anti-GAPDH (2118, Cell Signaling Technology), and horseradish peroxidase-conjugated anti-rabbit (7074, Cell Signaling Technology). Immunoblot band intensities of AR and GAPDH were quantified by ImageJ software, and the level of AR protein expression was normalized to the GAPDH values.

### Enzyme-Linked Immunosorbent Assay (ELISA)

ELISA to detect the AR protein was performed using a human AR ELISA kit (LS-F4505, LSBio) according to the manufacturer's manual. In brief, HFDP cells (5×10⁴ cells/well) were collected and lysed using cell lysis buffer (Cell Signaling Technology), and then, 100 μl of cell lysate was added to each well for 1 hr at 37° C. After incubation, a biotin-conjugated detection antibody (detection reagent A) was added that bound to the captured antigen. An avidin-horseradish peroxidase conjugate (detection reagent B) that binds to biotin was then added. The TMB substrate reacts with the HRP enzyme, resulting in color development. A sulfuric acid solution (stop solution) terminated the color development reaction, and the optical density (OD) of each well was measured at a wavelength of 450 nm. The OD of samples was computed with an OD standard curve generated using known standard AR concentrations (0.313˜20 ng/ml) to determine its AR concentration. The incubation and washing steps were performed according to the supplier's manual. Absorbance at 450 nm was monitored with a FLUOstar Omega microplate reader (BMG Labtech).

### Cell Viability Assay

For the cell viability assay, HaCaT and HFDP cells were seeded at a density of 4×10³ cells/well in a 96-well plate and then treated with the indicated conditions for 96 h. After incubation, 10 μl of WST (water soluble tetrazolium salt) reagent (EZ-Cytox, DoGen) was added to each well for 30 min at 37° C. Absorbance at 450 nm was monitored with a FLUOstar Omega microplate reader (BMG Labtech).

### Immunofluorescence (IF)

Plucked hairs from healthy donors were obtained from the occipital area of the scalp. After incubation, hair follicles were washed three times with 0.05% Tween-20 (Sigma) in PBS for 5 min and permeabilized in 0.1% Triton X-100 (Sigma) in PBS for 1 min. Hair follicles were fixed in 4% formaldehyde (Sigma) in PBS for 20 min, placed on a cryotray and covered with OCT compound (Tissue-Tek). The mold was slowly placed in liquid nitrogen until the entire tissue block was completely frozen. The frozen tissue blocks were sectioned at 10 μm using a cryotome (Leica Biosystems). Hair follicle sections were incubated overnight at 4° C. with an anti-AR antibody (ab133273, Abcam). The sections were subsequently incubated with anti-rabbit Fluor 568 (A11011, Invitrogen) secondary antibody for 1 hr at room temperature. After at least three washes, the sections were counterstained with DAPI (D9542, Sigma-Aldrich) and mounted in Immu-Mount solution (Thermo Fisher Scientific). All incubations were conducted in dark and humid chambers. The fluorescence signal was visualized using a confocal microscope (LSM880, Carl Zeiss) at excitation wavelengths of 568 nm (Alexa Fluor 568) and 405 nm (DAPI). At least three fields per section were analyzed. ZEN software (Zeiss) was used to analyze images of the desired area to measure the intensity on a pixel-by-pixel basis and calculate the mean intensity automatically.

### Statistical Analysis

All data are presented as the mean±standard deviation (SD), and the number of samples is indicated in each figure legend. The statistical significance of differences was assessed using the two-sided Student's t test. The results shown are representative of at least three independent experiments. Significance was denoted as *p<0.05, ** p<0.01, and *** p<0.001.

Clinical Study I: Low-Dose SAMiRNA-AR68 (0.5 mg/ml) Treatment Three Times Per Week

Clinical study I was a double-blind, randomized, placebo-controlled study in accordance with the Korea Ministry of Food and Drugs Safety (MFDS) Guidelines on the Efficacy Study of Cosmetics for Hair Loss Relief (July 2018). The study was approved by the Institutional Review Board of the local committee, Seowon Skin Research Center, and was conducted on Oct. 9, 2020 (IRB No. 1040820-202009-HR004-02) and all experimental protocols were carried out in accordance with relevant guidelines and regulations. All subjects provided written informed consent for publication of identifying information/images in an online open-access publication prior to study participation. Summary information of the study is listed and available at CRIS Registration No. KCT0005501: https://cris.nih.go.kr/cris/search/detailSearch.do/20301. Subjects were selected on the basis of inclusion and exclusion criteria and consisted of 48 Korean males and females ranging from 22 to 53 years of age. Male subjects were diagnosed with more than the M1, C1 and U1 range as the basic type and more than the VI and F1 range as the specific type according to the basic and specific (BASP) classification⁴⁹. Female subjects were diagnosed with more than the F1 and L1 ranges according to the BASP and Ludwig classification, respectively. The exclusion criteria were participation in a previous study within 6 months, prior surgical correction of scalp hair loss, use of topical minoxidil or finasteride within 6 months, and other skin diseases of the scalp, including severe seborrheic dermatitis, psoriasis, lichenoid eruption or other scalp infections. Alterations in hair styling and dyeing of the hair were not allowed during the study.

Clinical Study II: High-Dose SAMiRNA-AR68 (5 mg/ml) Treatment Once a Week

The second clinical study was conducted as a double-blind, randomized, placebo-controlled study in accordance with the Korea MFDS Guideline on the Efficacy Study of Cosmetics for Hair Loss Relief (July 2018). A summary of the study details is available through the following link: https://cris.nih.go.kr/cris/search/detailSearch.do/20367 (CRIS Registration No. KCT0005618). Clinical study II was approved by the Institutional Review Board of the local committee, Ellead Skin Research Center, and was conducted on Jul. 9, 2020 (IRB No. 200804T001) and all experimental protocols were carried out in accordance with relevant guidelines and regulations. All subjects provided written informed consent for publication of identifying information/images in an online open-access publication prior to study participation. The subjects were selected on the basis of inclusion and exclusion criteria and consisted of 60 Korean males and females ranging from 22 to 54 years of age. The AR68 high-dose (5 mg/ml) formulation was the same as the composition used in clinical study I, and randomly blinded products were applied once per week after shampooing. The measurement and analysis of hair density and total hair counts were conducted using the same protocol as described for clinical study I. The total hair count was measured using a Folliscope® 5.0 phototrichogram system (LeadM). Statistical analysis was also performed using the same program, statistical software SPSS statistics version 26.0 (IBM), as in clinical study I.

### SAMIRNA-AR68 Formulation and Treatment

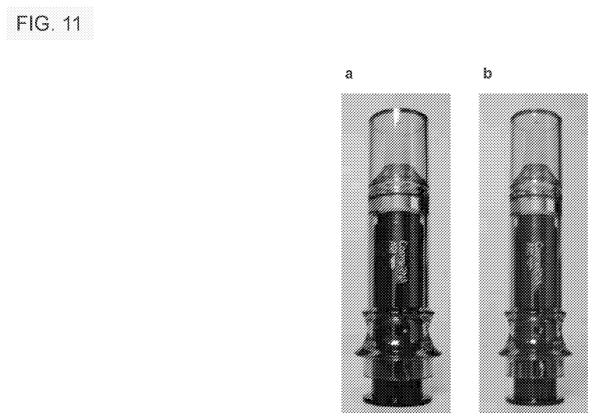

SAMiRNA-AR68 was formulated as a hair tonic aqueous solution with ethanol (15%, v/v), niacinamide (1% w/v), betaine (1% w/v), biotin (0.02% w/v) and buffer. The AR68 hair tonic was packaged in a piston-type bottle with a silicon adaptor for massage (FIG. 11). The placebo was prepared in the same manner as the formula except for the addition of AR68 and packaged in the same bottle. In clinical study I, randomly selected products (AR68, 0.5 mg/ml) were used three times per week after shampooing, whereby the scalp was massaged for 5 minutes after application. In clinical study II, randomly selected products (AR68, 5 mg/ml) were used once a week after shampooing, whereby the scalp was massaged for 5 minutes after application. Product usage and compliance were monitored for each subject by reviewing subject diaries and weighing the returned test products at each scheduled visit (weeks 8, 16 and 24).

### Measurement of Hair Density and Total Hair Count

All subjects placed their head into a hair photograph device (Canfield Scientific), and photographs were taken at fixed distance, angle and lighting with an EOS Rebel T6i digital camera (Canon). Global evaluation was performed by comparing clinical images at baseline with those at 8, 16 and 24 weeks after treatment with the test product. The investigator qualitatively assessed clinical images on a 7-point scale (−3, marked decrease; −2, intermediate decrease; −1, slight decrease; 0, no change; +1, slight increase; +2, intermediate increase; and +3, marked increase).

For evaluation of total hair counts, the hair was clipped approximately 2 mm after a red spot tattoo in the evaluation area (1 cm²) of the hair loss region (forehead hairline or vertex). The total hair counts were assessed using a Folliscope® 2.8 phototrichogram system (magnification 14 times, LeadM) at baseline and at 16 weeks and 24 weeks after treatment. The total hair count (number/cm²) was calculated as the number of hairs within an area.

### Subject Self-Assessment Questionnaires

Subject self-assessment questionnaires on efficacy and safety at 8 weeks, 16 weeks, and 24 weeks after using the product were collected.

### Safety Assessments

Dermatologists observed and assessed the subjects for the occurrence of objective irritation, such as edema, itching, and burning. The details, including start and end dates of occurrence, degree of severity, treatment, and causal relationship with the test product, were recorded on the form. If any adverse reaction occurred, the dermatologist's assessment of the adverse reaction was further conducted in accordance with the standard operating procedures for adverse reactions.

### Statistical Analysis

Data are expressed as the mean and a changing rate between the baseline value and the value obtained at each time point. All statistical analyses were performed using the SPSSR package (IBM). For photographic assessment of hair density, statistical analysis for variables for comparison between time points or groups was conducted using the Wilcoxon signed-rank test and Mann-Whitney U test (p<0.05). For analysis of total hair counts, the significance of a difference between time points was calculated using the paired t test, with p<0.05 as the significance level.

### 7.8. Clinical Study III: Monocentric Randomized Double-Blind Placebo Controlled Clinical Trial of High-Dose SAMiRNA-AR68 (5 mg/ml) Over a Period of 6 Month in Total Including Intermediate Analysis after 4 Months)

### General Characteristics of the Subjects

The study was carried out with 40 female and 80 male subjects in the age of 23-72 years, according to the inclusion and exclusion criteria.

Inclusion criteria:
18 years and older
Female and male healthy volunteers
Skin type: any
Hair loss (alopecia), Norwood scale stadium at least III or more in-creased hair loss, respectively Ludwig scale stadium at least I-3 or more increased hair loss
Written informed consent of the subjects or legal guardian is available

### Exclusion Criteria:

Severe or chronic skin inflammations
Severe internal or chronic diseases
Taking of drugs that may interfere with skin reactions (glucocorticoids, antiallergics, topical immune modulators, etc.)
Application of active substance-containing products and care products 7-10 days before the start of the test
Severe allergies or any serious side effects of cosmetic preparations ever occurred
Sun baths or solarium visits during the study
Known neoplastic disease
Pregnancy and breast-feeding

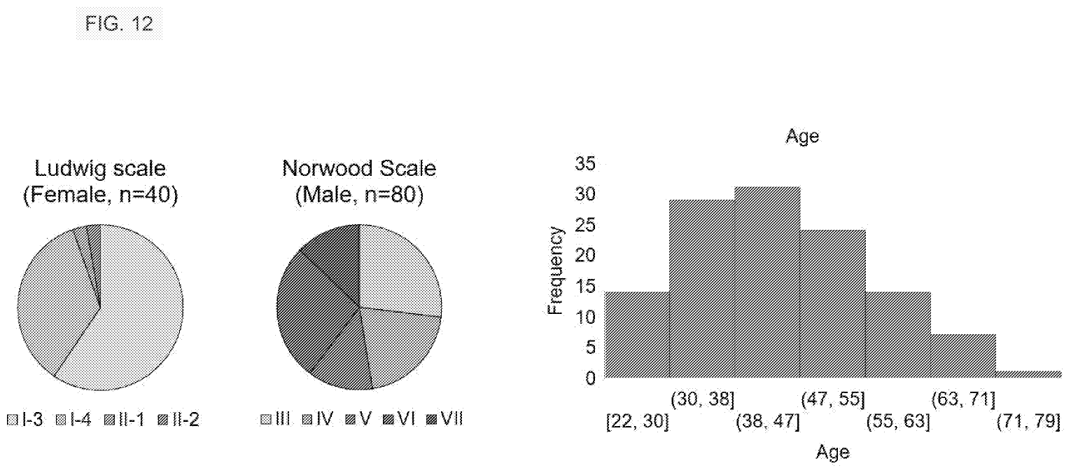

Demographics of the subjects are summarized in FIG. 12. A total of 115 male and female subjects were randomly assigned to an SRN-021 treatment group (test group) or placebo group as provided in the below table 7. The test group was further divided into three groups-subjects in the first group (28 subjects) were treated with SRN-021 (5 mg/ml) once a week, subjects in the second group (28 subjects) were treated with SRN-021 (5 mg/ml) once every two week, and subjects in the third group (29 subjects) were treated with SRN-021 (5 mg/ml) once a month. The test product tonic was applied on scalp at areas with less dense hair.

TABLE 7

| | | Dosage | | |
|---|---|---|---|---|
| Group | Trial Arms | (1 mL) | Frequency | Subject # |
| I | CosmeRNA | 5 mg/mL | 4/mo (Weekly) | 28 |
| II | CosmeRNA | 5 mg/mL | 2/mo | 28 |
| III | CosmeRNA | 5 mg/mL | 1/mo (Monthly) | 29 |
| IV | Placebo (Saline) | N/A | 4/mo (Weekly) | 30 |
| | Total | | | 115 |

### Analysis of Hair Density and Total Hair Counts

The special camera and software (version 4.0.10.102) of the TrichoScan HD system (DermoScan GmbH, Ger-many) were used to evaluate the density of hair (terminal and vellus hair) as well as the status of all analyzed single hairs concerning anagen or telogen stage. Further the thickness of hair was determined by the TrichoScan system and expressed as median value of all analyzed hairs beside expression as mean value of all analyzed hairs. The area on the scalp of a single measurement by the TrichoScan HD system was fixed to 0.59 cm², corresponding to a circle with 0.87 cm in diameter. The following pretreatment of the analyzed area on the scalp was carried out:

On the scalp of every subject one area with 2 cm in diameter was located by a dot mask in the transition zone between alopecia and regularly hirsute scalp. The hairs were threaded through the hole of the mask and roughly shortened with a pair of scissors. Further the shortened hairs were shaved to an even length of 0.8 mm by a Moser shaver (TrichoScan Edition). For this purpose, the razor was moved in 90° angle to the scalp without pressure, resulting in a shaved area with 2 cm in diameter.

The hair dye (Goldwell Topchic 2N) was applied by a wooden spatula on the shortened hairs 3 days after shaving. In detail the same amount of the developer (creme oxide) was added to the hair dye (1:1 mixture). Hair dye and developer were mixed thoroughly until they reached a creamy consistency. The dye mixture was applied to the shaved area on scalp of the subject and remained there for 15 minutes. After this incubation time the dye mixture was removed coarsely with a swab and the area was cleaned very carefully with an alcoholic tincture (e.g. Kodan spray) and a soft swab.

The camera of the TrichoScan HD system was used to record the image files at scheduled times of analysis. The scalp was moistened thoroughly (Kodan spray) before recording of images in the prepared area. Attention was paid to record the images without air bubbles and without surrounding hairs. By this procedure the parameters density of terminal and of vellus hair [1/cm²] as well as portion of anagen hair [%] and telogen hair [%] were quantified, using the TrichoScan HD software. Additionally portions of terminal and of vellus hair [%] as well as thickness of hair [μm] were determined. In this way 8 records (Trichogramms) were prepared in average per subject and per time of analysis.

The quantity of out fallen hair was determined as following: After washing the wet hair was combed intensively with a provided standardized comb by the subject, placing the head above a white DIN A4 paper sheet. This resulted in falling of hair on the paper sheet. The out fallen hair were counted by a qualified study nurse. This procedure was executed at scheduled times of analysis.

In addition to out fallen hair, criteria such as Anagen Hair (%), Density Terminal Hair [1/cm²], Density VellusHair [1/cm²], and Hair Thickness [μm] were measured over a period of 6 month in total including intermediate analysis after 4 months.

### Dermatological Examination Results

Dermatological examinations were carried out according to clinical-dermatological evaluation criteria. All subjects exhibited healthy skin in the test area before, during and after the application period. No pathological skin lesions were found in any form. Neither interruption of test product application, nor reduced product application, decided by a dermatological specialist, occurred in any case. Not any treatment by a dermatological specialist was necessary. The product SAMiRNA-AR68 was very well tolerated and did not induce dermatological relevant skin changes on any subject.

### Analysis of Total Hair Counts and Photographic Assessments

Trichoscan analyses were carried out in the prepared area of every subject's scalp at indicated times. Percentage of anagen hair was determined by 8 repetitive recordings in mean value over all subjects, placing the camera head on 8 different positions in the prepared scalp area and using the Trichoscan HD software for evaluation of recordings. The mean values of all repetitive records per subject were analyzed.

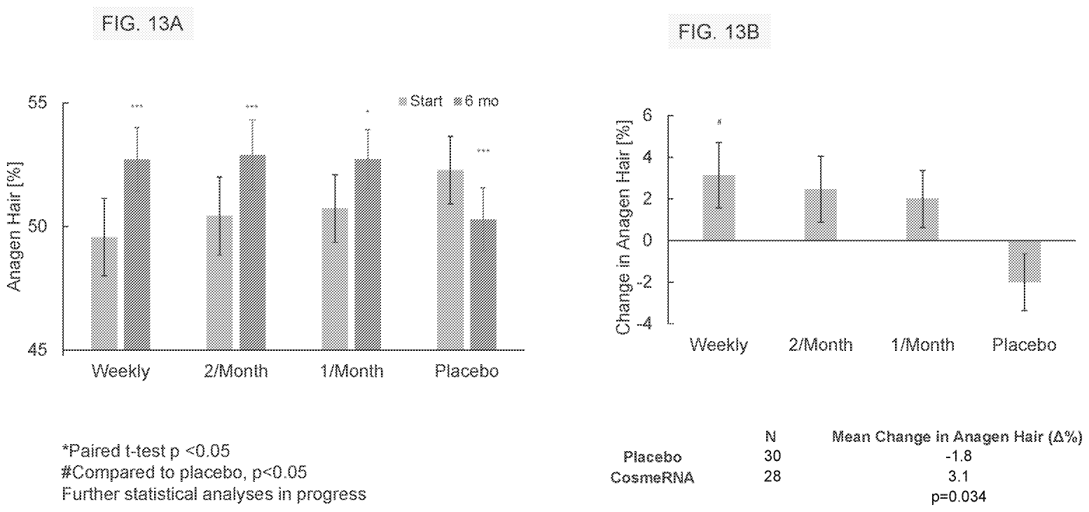

The ratio of anagen hair (%) was calculated in each placebo or treatment group (weekly, 2/month, 1/month) before the treatments (Start) and after the treatment for 6 months (6 mo) as provided in FIG. 13A. It shows that SAMiRNA-AR68 increased percentage of anagen hair in all treatment groups after 6 months of administration. Further, the changes in anagen hair percentages after treatment with SAMiRNA-AR68 for 6 months were calculated and provided in FIG. 13B. Anagen hair (Change in %) increased with weekly treatment of SAMiRNA-AR68 by 5% compared to Placebo.

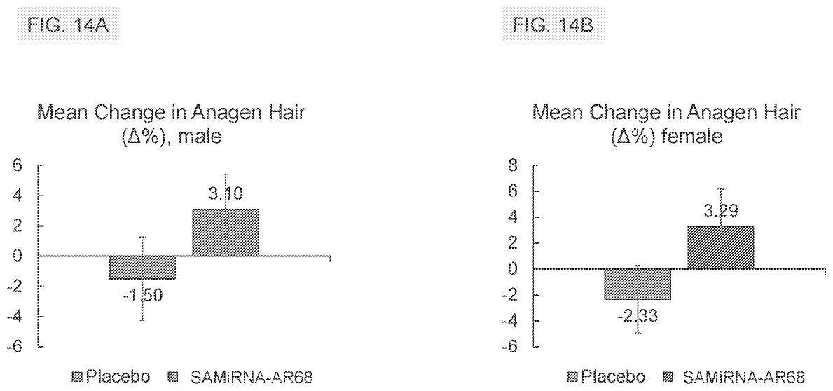

The changes in anagen hair percentages were further analyzed in males (FIG. 14A) and females (FIG. 14B). In men, Anagen Hair (Change in %) increased with SAMiRNA-AR68 by over 4.6% compared to Placebo. In women, Anagen Hair (Change in %) increased with SAMiRNA-AR68 by over 5.6% compared to Placebo.

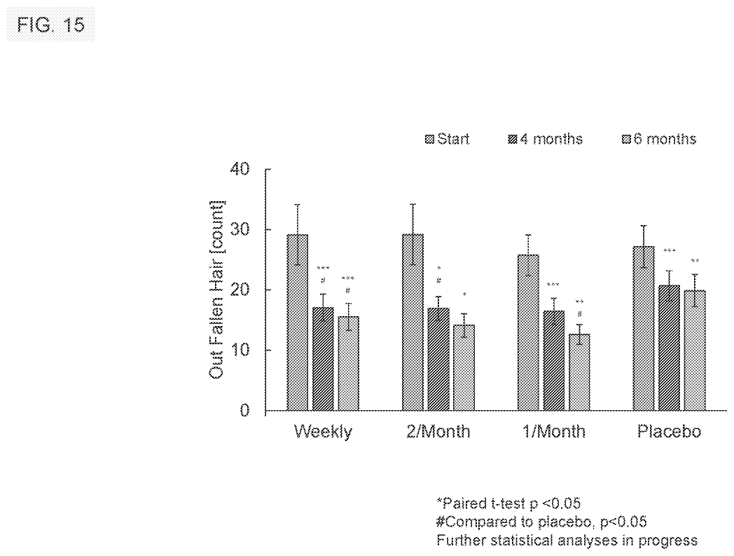

The efficacy of SAMiRNA-AR68 was further tested by measuring reduction of hair loss by counting the out fallen hair after washing and combing by a standardized comb. The results are provided in FIG. 15. The result shows significant decrease in the number of fallen hairs after treatment with SAMiRNA-AR68.

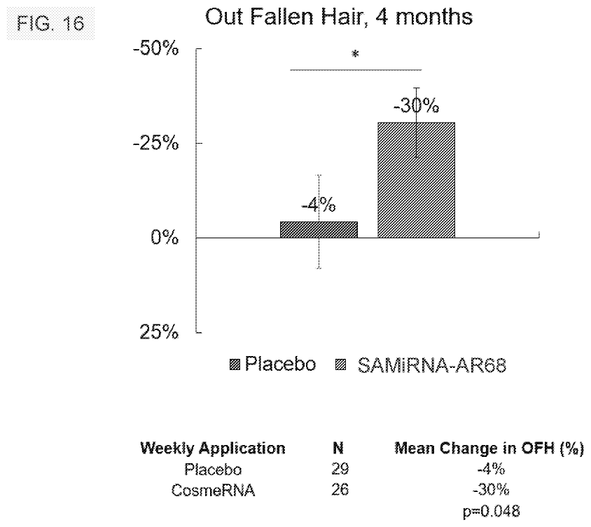

The subject treated with SAMiRNA-AR68 weekly showed about 30% reduction of fallen hair after 4 months. As shown in FIG. 16, overall fallen hair decreased with SAMIRNA-AR68 by average 26% compared to Placebo at four months with response rate of 80%.

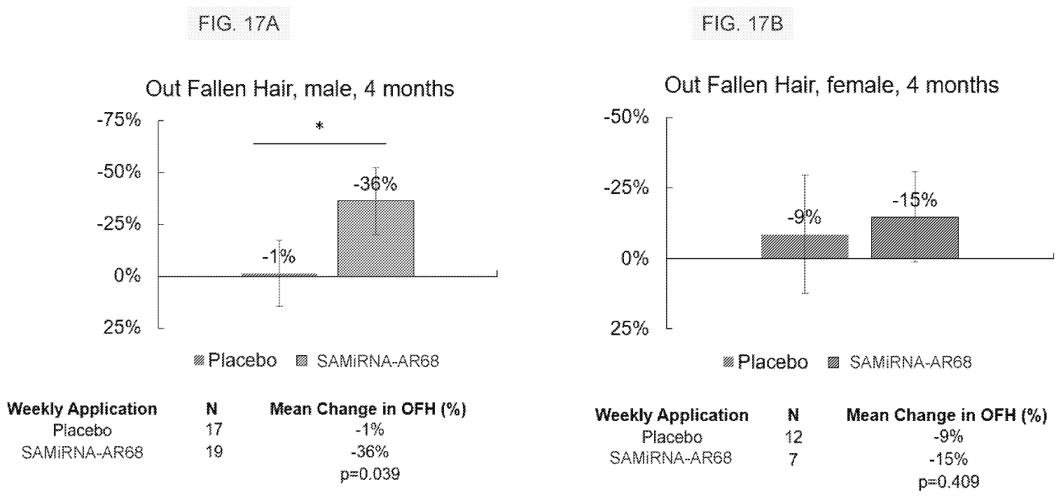

The effect was also compared between males and females. In men (FIG. 17A), Out Fallen Hair decreased with SAMiRNA-AR68 by 35% compared to Placebo at four months. In women (FIG. 17B), Out Fallen Hair decreased with CosmeRNA by 16% compared to Placebo at four months. The effect was more significant in men than in women.

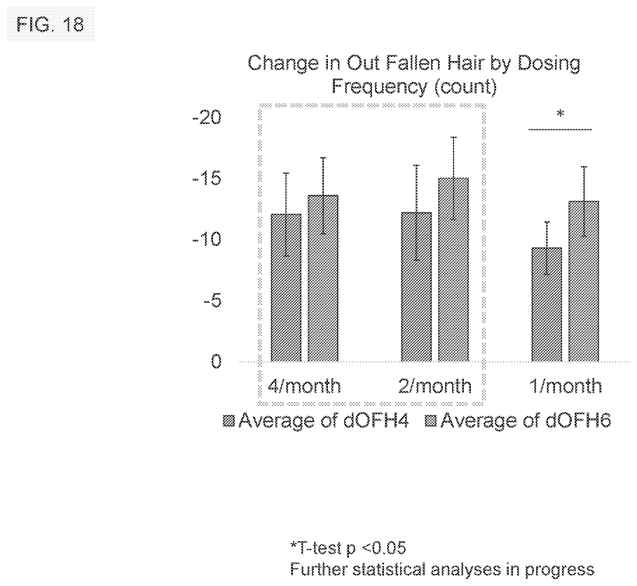

Changes in the Out Fallen Hair were compared between different dosing frequencies—weekly (4/month), biweekly (2/month) and monthly (1/month). The average changes in each group were measured after 4 months and after 6 months from initiation of the SAMiRNA-AR68 treatment. As provided in FIG. 18, weekly or biweekly (2/month) application reached 12-13 net gain of hair by 4 months from start of the regimen. Monthly application achieved comparable net gain of hair by 6 months. In the group of monthly treatment with SAMiRNA-AR68, there was significant difference in the effect between 4-month and 6-month. However, the difference between 4-month and 6-month was not significant in the groups treated with SAMiRNA-AR68 weekly or biweekly. (FIG. 18 and Table 8)

TABLE 8

| | | | |
|---|---|---|---|
| Arm | N | Mean | Std. Error Mean |

| | | | | |
|---|---|---|---|---|
| A1 | 4 mo | 28 | −12.1 | 6.8 |
| | 6 mo | 28 | −13.6 | 6.2 |
| A2 | 4 mo | 28 | −12.2 | 7.7 |
| | 6 mo | 28 | −15.0 | 6.7 |
| A4 | 4 mo | 29 | −9.3 | 4.3 |
| | 6 mo | 29 | −13.1 | 5.7 |

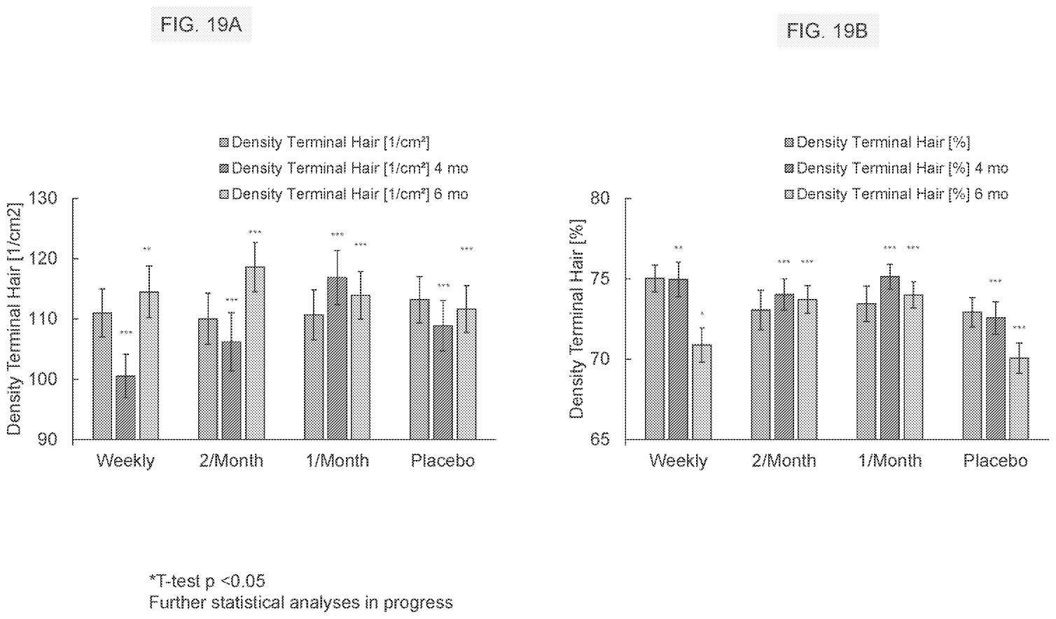

The density of terminal hair was investigated by Trichoscan technique as another way to test efficacy of SAMiRNA-AR68. FIGS. 19A and 19B show the density of terminal hair measured as 1/cm² (FIG. 19A) or % (FIG. 19B) in different dosing frequency groups-weekly (4/month), biweekly (2/month) and monthly (1/month), before or 4 or 6 months after treatment with SAMiRNA-AR68. The data show density of terminal hair tends toward increasing values in the treatment groups compared to the placebo group.

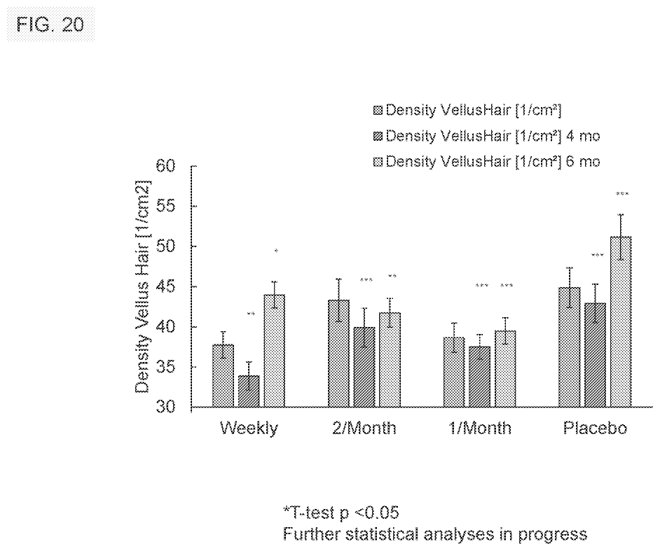

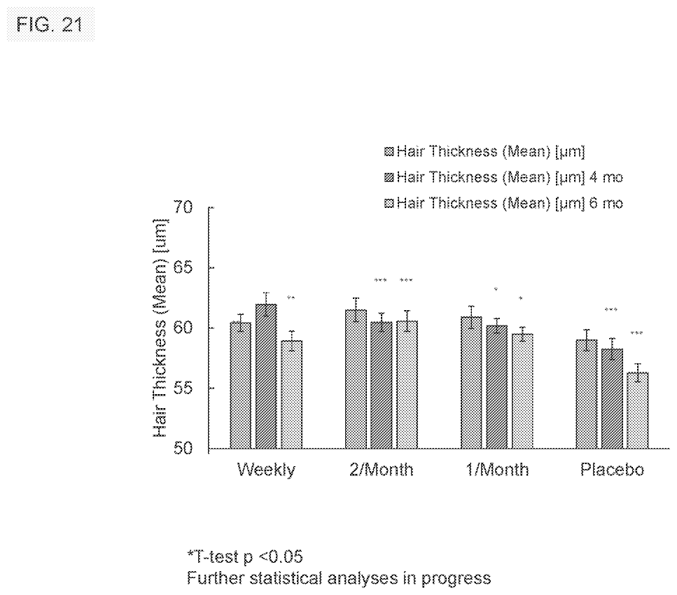

The density of vellus hair and the hair strand thickness were also measured in each group and the results are provided in FIG. 20 and FIG. 21, respectively.

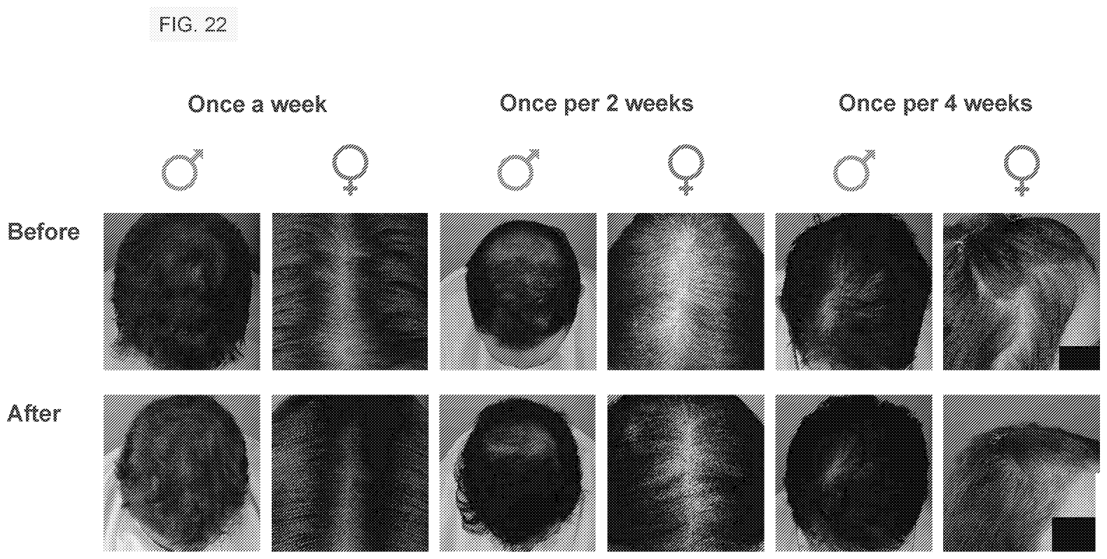

The hair improvement effects of SAMiRNA-AR68 were significant and visible improvements were observed after 6 months of use (FIG. 22). And there was no adverse skin reaction was observed throughout the study.

### 7.9. The Stability of SAMiRNA-AR68 Formulation

SAMiRNA-AR68 was formulated as a hair tonic aqueous solution with the following compositions (Table 9). The formulation was used in Clinical Study III and stability of the formulation was assessed and evaluated. The formulation is a colorless or pale yellow, transparent liquid. The pH-value is 5.12 and within the range of pH 4.10 to 6.10. The difference between the main peak obtained from the test solution of Phosphate Buffered Saline (and) Stearyldisulfidehexyl sh-DNA-2 PEG-45/sh-RNA-1 (SAMiRNA-AR68) and the retention time of the main peak obtained from the standard solution is within the range of +2.5%. The particle size (mean value) is 109 nm and within the specification of <200 nm.

TABLE 9

| | % of ingredient | | % of ingredient | |
|---|---|---|---|---|
| RM Name | in RM | INCI | in FP | Function |
| cosmerna-68 | 98.50% | Phosphate buffered saline | 33.3 | Solvent |
| | 1.50% | Stearyldisulfidehexyl sh- | | Hair |
| | | DNA-2 PEG-45/sh-RNA-1 | | conditioning |
| | | (SAMiRNA-AR68) | | |
| D.I water | 100% | Water | 46.38 | Solvent |
| Denatured Ethanol | ≥95% | Ethanol | 15 | Solvent |
| | ≤5% | Water | | |
| D-Panthenol | 98% | Panthenol | 0.2 | Hair |
| | ≤1% | Water | | conditioning |
| | ≤1% | Is Aminopropanol | | |
| GENENCARE | 100% | Betaine | 1 | Humectant |
| OSMS BA | | | | |
| Biotin | 100% | Biotin | 0.02 | Hair |
| | | | | conditioning |
| Niacinamide | 99% | Niacinamide | 1 | Smoothing |
| Citric acid | ≥99.7% | Citric Acid | 0.02 | Buffering |
| Menthol Crystals | ≥99% | Menthol | 0.05 | soothing |
| Trisodium citrate | ≥99% | Sodium Citrate | 0.03 | Buffering |
| Dihydrate | | | | |
| 1,3-Butylene glycol | ≥99.5% | Butylene Glycol | 3 | Humectant |

For the evaluation of the chemical and physical stability, a stability test was performed with the formula. The results (pH value, weight loss and total plate count) showed that the product is stable after storing it 3 months (40° C., 75% RH). As a rule of thumb, “storing a sample at 40° C. for a period of one month corresponds to an actual shelf life of 10-12 months at room temperature. This indicates that for an expected minimum shelf life of 30 months, storage for a period of 3 months at 40° C. is or must be carried out as a rule.” (from: Praxishandbuch Kosmetische Mittel 19 10 29, II.7 Mikrobiologie kosmetischer Mittel, S. 12).

### 7.10. DNA-RNA Heteroduplex with Phosphorothioate Modifications

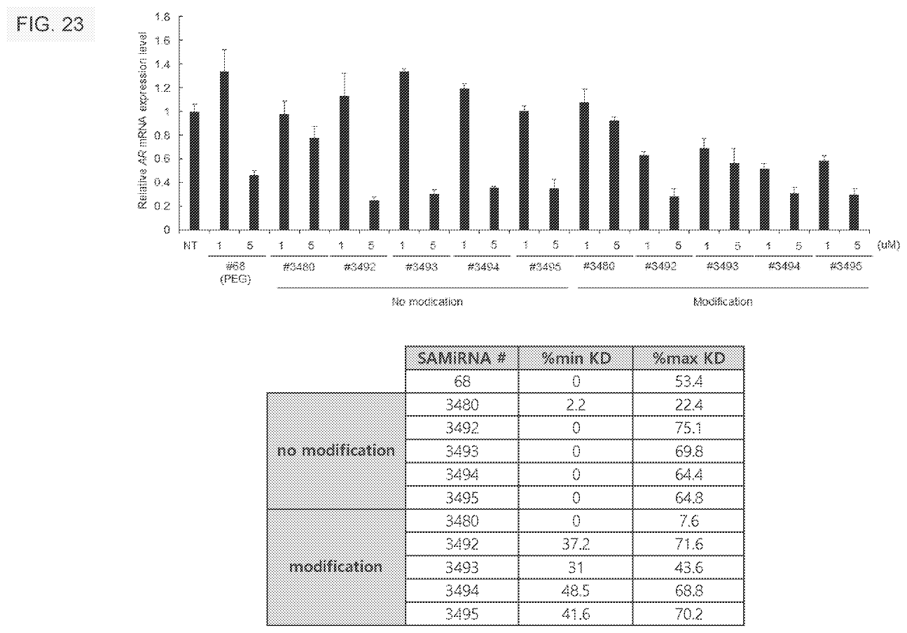

Several variants of SAMiRNA-AR68 (#3480, 3492, 3493, 3494, 3495) were generated by changing the DNA/RNA strand sequences. Additionally, the variants were further modified by using phosphorothioate nucleotides in the synthesis of the DNA/RNA strand. Each of the variants with and without phosphorothioate modifications was tested for its knockdown efficacy in human follicle dermal papilla cells. Human follicle dermal papilla (HFPD) cells were treated with the SAMiRNAs for 72 hrs. Total RNA extracts were subjected to RT-qPCR assays, and AR mRNA expression level was normalized to that of the RPLP0 gene. The results are provided in FIG. 23. The numerical data are also provided in the table (FIG. 23).

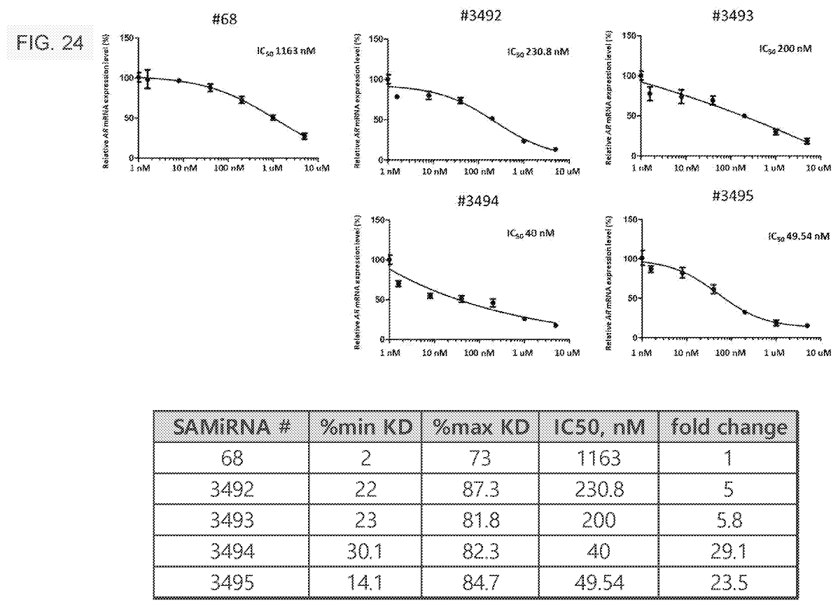

The results show in general that SAMiRNAs with phosphorothioate modifications have stronger inhibitory effects on the AR mRNA expression. Further, the assay allowed identification of AR-68 variants (e.g., #3494 and 3495) having stronger inhibitory effects on AR mRNA expression than other variants. The half-maximal inhibitory concentration (IC50) of the variants was further tested and analyzed. The data provided in FIG. 24 show that #3494 (SRN-021) has significantly greater inhibitory effects (about 30-fold) against AR expression compared to SAMIRNA AR-68. The half-maximal inhibitory concentration (IC50) of #3494 (SRN-021) was 40 nM whereas IC50 of SAMiRNA-AR68 was 1163 nM.

The #3494 variant (SRN-021) and SAMiRNA-AR68 have multiple differences as summarized in the below table 10. SRN-021 includes a modified RNA strand comprising the sequence of SEQ ID NO: 2 with phosphorothioate modifications (UUGGAGCCAUCCAAAC*U*C*U, SEQ ID NO: 32), wherein each nucleotide of UCU at 3′-end of SEQ ID NO: 2 is a phosphorothioated nucleotide. Additionally, SAMiRNA-SRN-021 is different from SAMiRNA-AR68 by having hexaethyleneglycol (HEG) covalently attached to 3′-end of the DNA strand.

TABLE 10

| | SAMiRNA-AR68 | SAMiRNA-SRN-021 |
|---|---|---|
| Hydrophilic | PEG2000 | HEGX4 |
| chain | | |
| Modification | None | Phosphorothioate |
| Sequence | Sense: | Sense: |
| | GAGTTTGGATGGCTCCAAA | AGAGTTTGGATGGCTCCAA |
| | (SEQ ID NO: 3) | (SEQ ID NO: 1) |
| | Antisense: | Antisense: |
| | UUUGGAGCCAUCCAAACUC | UUGGAGCCAUCCAAAC*U*C*U |
| | (SEQ ID NO: 4) | (SEQ ID NO: 2 with phosphorothioate |
| | | modifications at *U*C*U) |

### 7.11. SAMiRNA-SRN-021 Decreased AR mRNA and Protein Levels in Skin in Mice

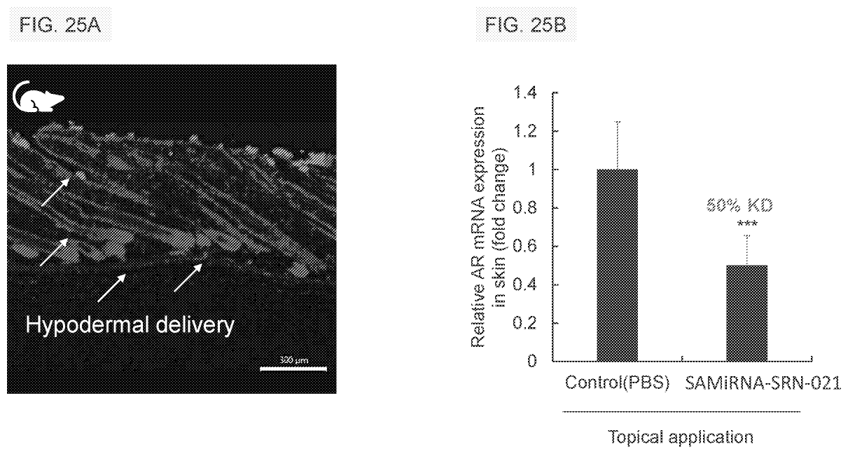

To evaluate SAMiRNA nanoparticle delivery and AR inhibitory effect of SAMiRNA-SRN-021, fluorescein (Cy5)-labeled SAMiRNA-SRN-021 was applied to the skin in mice. The fluorescent image in FIG. 25A shows that SAMiRNA-SRN-021 delivered RNAi through appendageal (intercellular) penetration. This suggests that SAMiRNA-SRN-021 provides effective hypodermal delivery of the RNAi.

Additionally, SAMiRNA-SRN-021 effectively knockdown expression of AR mRNA in the skin. The in vivo knockdown achieved through the topical delivery was about 50%. (FIG. 25B)

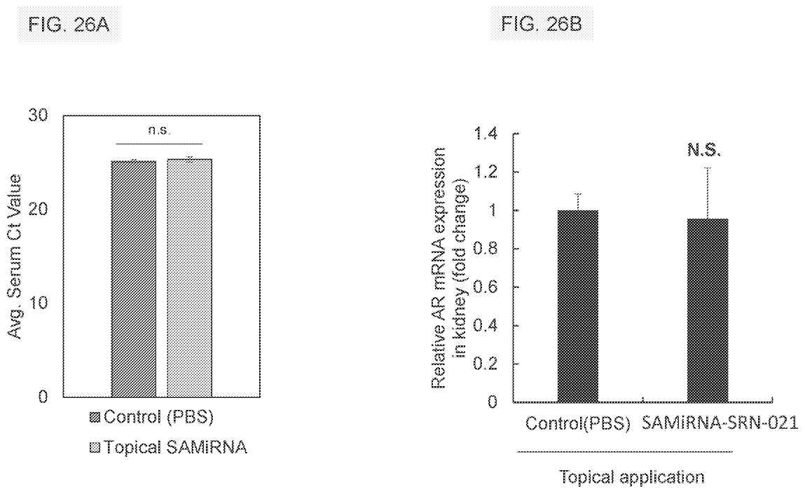

SAMiRNA-SRN-021 topically applied to the skin remained local without systemic delivery. Serum from the mice treated with SAMiRNA-SRN-021 was collected and tested for the presence of SAMiRNA by RT-qPCR amplification using stem-loop primers. A 10-fold serial diluted serum was detected at 7.99 to 23.39 Ct (cycle threshold). In the test, 23.39 Ct is the lowest limit that the stem-loop RT-PCR primer set for SAMiRNA-SRN-021 could detect (LOD, limit of detection). SAMIRNA was not detected in the serum as provided in FIG. 26A. Additionally, AR mRNA expression was measured in kidney to test whether there is any modulation of AR expression in distal organs. There was no change in AR mRNA expression detected in kidney (FIG. 26B), suggesting that there is no systemic exposure of SAMiRNA-SRN-021 after topical application.

8. SEQUENCE LISTING

| SEQ ID NO | SEQUENCE |
|---|---|
| 1 | AGAGTTTGGATGGCTCCAA |
| 2 | UUGGAGCCAUCCAAACUCU |
| 3 | GAGTTTGGATGGCTCCAAA |
| (AR68 sense) | |
| 4 | UUUGGAGCCAUCCAAACUC |
| (AR68 anti sense) | |
| 5 | ATGTACAGTCTGTCATGAA |
| (AR109 sense) | |
| 6 | TTGCCCATTGACTATTACT |
| 7 | AATGAGTACCGCATGCACA |
| 8 | GAAAGTCAAGCCCATCTAT |
| 9 | ATTGATGTACAGTCTGTCA |
| 10 | TGCTCGATGTGGACGAAGA |
| 11 | ACATGGGAGTTGTTGGATT |
| 12 | AGCTAACATTGAGCTTCAA |
| 13 | AGGCTGACTGTCTACGAAT |
| 14 | TGATCAGTTAACTAAAAGT |
| 15 | TTTACTAGTTCAAGACAGA |
| 16 | CTAGTTCAAGACAGATGAA |
| 17 | GAATGACCCACGCAAAAAA |
| 18 | TTGTACACGTGGTCAAGTGG |
| 19 | TGGAGTTGACATTGGTGAAGG |
| 20 | TGCCATTGCCCCATGTGAAG |
| 21 | AGCTGCACATCACTCAGGATT |
| 22 | CTGAGCTCGCCAGTGAAAT |
| 23 | CTGTAGTGGTGGTCGGAGA |
| 24 | AGATGCAATAACCACCCCTG |
| 25 | TGCGCAGAATGAGATGAGTT |
| 26 | CTGTAGCCCATGTTGTAGCA |
| 27 | GGTTATCTCTCAGCTCCACG |
| 28 | GAATGTCCAACGCAAAGCAA |
| 29 | ACCTCGAAACAGCATCTGAC |
| 30 | GTGTTTGACGGCATCCCACC |
| 31 | TAGGCTTCAGACGCACGACC |
| 32 | UUGGAGCCAUCCAAAC*U*C*U, where |
| | * indicates phosphorothioate |
| | modification of the following |
| | nucleotide |

### 9. EQUIVALENTS AND INCORPORATION BY REFERENCE

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

What is claimed is:

1. A DNA-RNA heteroduplex, comprising:
a. a DNA strand having the sequence of SEQ ID NO: 1 or 3,
b. an RNA strand having the sequence of SEQ ID NO: 2 or 4, or a modified RNA strand comprising the sequence of SEQ ID NO: 2 or 4 with one or more modifications,
c. stearyldisulfidehexyl (C₆—S—S—C₁₈) covalently attached to 5′-end of the DNA strand; and
d. hexaethylene glycol (HEG) or polyethylene glycol (PEG) covalently attached to 3′-end of the DNA strand,
wherein the DNA strand and the RNA strand or the modified RNA strand form a DNA-RNA hybrid.

2. The DNA-RNA heteroduplex of claim 1, wherein the DNA strand has the sequence of SEQ ID NO: 1 and the RNA strand has the sequence of SEQ ID NO: 2.

3. The DNA-RNA heteroduplex of claim 2, wherein the DNA strand has the sequence of SEQ ID NO: 3 and the RNA strand has the sequence of SEQ ID NO: 4.

4. The DNA-RNA heteroduplex of any one of claims 1-3, comprising HEG, wherein the HEG is hexaethyleneglycol-(—PO₃-hexaethyleneglycol) 3.

5. The DNA-RNA heteroduplex of any one of claims 1-4, comprising the modified RNA strand, wherein the modified RNA strand comprises the sequence of SEQ ID NO: 2 or 4 with one or more phosphorothioate modifications.

6. The DNA-RNA heteroduplex of claim 5, wherein the modified RNA strand comprises the sequence of SEQ ID NO: 2 and each nucleotide of UCU at 3′-end of SEQ ID NO: 2 is a phosphorothioated nucleotide.

7. A composition for hair improvement comprising the DNA-RNA heteroduplex of any one of claims 1-6.

8. The composition of claim 7, further comprising one or more humectants selected from betaine and butylene glycol.

9. The composition of claim 7 or 8, further comprising one or more hair conditioning or soothing ingredients selected from panthenol, aminopropanol, biotin, niacinamide, and menthol.

10. The composition of any one of claims 7-9, further comprising one or more buffering ingredients selected from citric acid and sodium citrate.

11. The composition of any one of claims 7-10, wherein 0.1-2% by weight of the composition is the DNA-RNA heteroduplex.

12. The composition of claim 11, wherein 0.25-1% by weight of the composition is the DNA-RNA heteroduplex.

13. The composition of claim 12, wherein 0.4-0.6% by weight of the composition is the DNA-RNA heteroduplex.

14. The composition of claim 13, wherein 0.4%, 0.45%, 0.5%, 0.55%, or 0.6% by weight of the composition is the DNA-RNA heteroduplex.

15. The composition of claim 11, wherein 1%, 1.25%, 1.5%, 1.75% or 2% by weight of the composition is the DNA-RNA heteroduplex.

16. The composition of any one of claims 7-10, wherein 1-20 mg/ml of the composition is the DNA-RNA heteroduplex.

17. The composition of claim 16, wherein 2.5-10 mg/ml of the composition is the DNA-RNA heteroduplex.

18. The composition of claim 17, wherein 4-6 mg/ml of the composition is the DNA-RNA heteroduplex.

19. The composition of claim 18, wherein 4 mg/ml, 4.5 mg/ml, 5 mg/ml, 5.5 mg/ml, or 6 mg/ml of the composition is the DNA-RNA heteroduplex.

20. The composition of claim 16, wherein 10 mg/ml, 12.5 mg/ml, 1.5 mg/ml, 1.75 mg/ml, or 2 mg/ml of the composition is the DNA-RNA heteroduplex.

21. The composition of any one of claims 7-20, comprising denatured ethanol, D-panthenol, betaine, biotin, niacinamide, citric acid, menthol crystals, trisodium citrate dihydrate, and 1,3-butylene glycol.

22. The composition of any one of claims 7-21, comprising 30-35% of phosphate buffered saline, 0.4-0.6% of the DNA-RNA heteroduplex, 45-50% of water, 14-15% of ethanol, 0.19-0.2% of panthenol, 1% of niacinamide, 0.02% of citric acid, 0.05% of menthol, 0.03% of sodium citrate, 3% of butylene glycol and less than 0.01% of aminopropanol.

23. The composition of claim 22, comprising 32.8% of phosphate buffered saline, 0.5-0.6% of the DNA-RNA heteroduplex, 46.3% of water, 14-15% of ethanol, 0.19-0.2% of panthenol, 1% of niacinamide, 0.02% of citric acid, 0.05% of menthol, 0.03% of sodium citrate, 3% of butylene glycol and less than 0.01% of aminopropanol.

24. The composition of any one of claims 7-23, wherein the composition is in the form of ointment, paste, gel, jelly, serum, aerosol spray, non-aerosol spray, foam, cream, lotion, solution, or suspension formulation.

25. The composition of any one of claims 7-24, having a pH between 5 and 7.5.

26. The composition of claim 25, having a pH between 5.5 and 6.5, between 6 and 6.5 or between 7 and 7.5.

27. The composition of claim 25, having a pH 6.29 or 7.02.

28. The composition of any one of claims 1-27, wherein the DNA-RNA heteroduplex forms a nanoparticle.

29. The composition of claim 28, wherein the nanoparticle has a mean diameter between 50 and 200 nm.

30. The composition of claim 29, wherein the nanoparticle has a mean diameter between 50 and 150 nm, between 75 and 125 nm, between 80 and 120 nm, between 90 and 110 nm, or between 100 and 110 nm.

31. A method of inhibiting androgen receptor expression, comprising applying the DNA-RNA heteroduplex of any one of the claims 1-6 or the composition of any one of claims 7-30 to a subject.

32. The method of claim 31, wherein the DNA-RNA heteroduplex or the composition is applied topically.

33. The method of claim 31 or 32, wherein the DNA-RNA heteroduplex or the composition is applied for hair improvement in the subject.

34. The method of claim 33, wherein the DNA-RNA heteroduplex or the composition is applied on the scalp of the subject.

35. The method of any one of claims 31-34, wherein the DNA-RNA heteroduplex or the composition is applied once a week, once per two weeks or once per four weeks.

36. The method of claim 35, wherein the DNA-RNA heteroduplex or the composition is applied for at least four months or at least six months.

37. The method of claim 35, wherein the DNA-RNA heteroduplex or the composition is applied once a week for at least four months.

38. The method of claim 35, wherein the DNA-RNA heteroduplex or the composition is applied once a week for four months.

39. The method of any one of claims 31-38, comprising applying the DNA-RNA heteroduplex or the composition once a week for four months, and applying the DNA-RNA heteroduplex or the composition less than once a week after the four months.

40. The method of claim 39, comprising applying the DNA-RNA heteroduplex or the composition once a week for four months, and after the four months applying the DNA-RNA heteroduplex or the composition once per 2 weeks, once per 3 weeks or once per 4 weeks.

41. The method of claim 39, comprising applying the DNA-RNA heteroduplex or the composition once a week for four months, and after the four months applying the composition once per 1 month, once per 2 months, once per 3 months, once per 4 months, once per 5 months, once per 6 months, or less.

42. The method of claim 39, comprising applying the DNA-RNA heteroduplex or the composition once a week for four months, and after the four months discontinuing application of the composition.

43. The method of any one of claims 31-42, wherein the subject has alopecia.

44. The method of any one of claims 31-42, wherein the subject has moderate androgenetic alopecia.

45. The method of any one of claims 31-44, wherein the subject has Norwood scale stadium at least III or more increased hair loss.

46. The method of any one of claims 31-45, wherein the subject has Ludwig scale stadium at least I-3 or more increased hair loss.

47. The method of any one of claims 31-46, wherein the composition applied to the subject comprises the DNA-RNA heteroduplex at 1-20 mg/ml.

48. The method of claim 47, wherein the composition applied to the subject comprises the DNA-RNA heteroduplex at 2.5-10 mg/ml.

49. The method of claim 48, wherein the composition applied to the subject comprises the DNA-RNA heteroduplex at 4 mg/ml, 4.5 mg/ml, 5 mg/ml, 5.5 mg/ml, or 6 mg/ml.

50. The method of claim 49, wherein the composition applied to the subject comprises the DNA-RNA heteroduplex at 4.5 mg/ml, 5 mg/ml, or 5.5 mg/ml.

51. The method of any one of claims 31-50, comprising applying 1-100 mg of the DNA-RNA heteroduplex once a week.

52. The method of claim 51, comprising applying 1-30 mg of the DNA-RNA heteroduplex once a week.

53. The method of any one of claims 31-50, comprising applying 5-10 mg of the DNA strand or the RNA strand once a week.

54. The method of claim 51, comprising applying 1-10 mg of the DNA strand or the RNA strand once a week.

55. The method of any one of claims 31-54, comprising applying 1-100 mg of the DNA-RNA heteroduplex per area scalp (cm²) once a week.

56. The method of claim 54, comprising applying 1-50 mg of the DNA-RNA heteroduplex per area scalp (cm²) once a week.

57. The method of claim 55, comprising applying 5-20 mg of the DNA-RNA heteroduplex per area scalp (cm²) once a week.

58. The method of claim 57, comprising applying 8-10 mg of the DNA-RNA heteroduplex per area scalp (cm²) once a week.

59. The method of any one of claims 31-54, comprising applying 0.5-5 ml of the DNA-RNA heteroduplex per area scalp (cm²) once a week.

60. The method of claim 59, comprising applying 1-2 ml of the DNA-RNA heteroduplex per area scalp (cm²) once a week.

61. The method of claim 60, comprising applying 1-1.5 ml of the DNA-RNA heteroduplex per area scalp (cm²) once a week.


SEQUENCE LISTING (32 sequences)

SEQ ID NO: 1 - DNA, 19 bp, synthetic construct
  source 1..19: mol_type = other DNA
agagtttggatggctccaa

SEQ ID NO: 2 - RNA, 19 nt, synthetic construct
  source 1..19: mol_type = other RNA
ttggagccatccaaactct

SEQ ID NO: 3 - DNA, 19 bp, synthetic construct
  source 1..19: mol_type = other DNA
gagtttggatggctccaaa

SEQ ID NO: 4 - RNA, 19 nt, synthetic construct
  source 1..19: mol_type = other RNA
tttggagccatccaaactc

SEQ ID NO: 5 - DNA, 19 bp, Homo sapiens
  source 1..19: mol_type = genomic DNA
atgtacagtctgtcatgaa

SEQ ID NO: 6 - DNA, 19 bp, Homo sapiens
  source 1..19: mol_type = genomic DNA
ttgcccattgactattact

SEQ ID NO: 7 - DNA, 19 bp, Homo sapiens
  source 1..19: mol_type = genomic DNA
aatgagtaccgcatgcaca

SEQ ID NO: 8 - DNA, 19 bp, Homo sapiens
  source 1..19: mol_type = genomic DNA
gaaagtcaagcccatctat

SEQ ID NO: 9 - DNA, 19 bp, Homo sapiens
  source 1..19: mol_type = genomic DNA
attgatgtacagtctgtca

SEQ ID NO: 10 - DNA, 19 bp, Homo sapiens
  source 1..19: mol_type = genomic DNA
tgctcgatgtggacgaaga

SEQ ID NO: 11 - DNA, 19 bp, Homo sapiens
  source 1..19: mol_type = genomic DNA
acatgggagttgttggatt

SEQ ID NO: 12 - DNA, 19 bp, Homo sapiens
  source 1..19: mol_type = genomic DNA
agctaacattgagcttcaa

SEQ ID NO: 13 - DNA, 19 bp, Homo sapiens
  source 1..19: mol_type = genomic DNA
aggctgactgtctacgaat

SEQ ID NO: 14 - DNA, 19 bp, Homo sapiens
  source 1..19: mol_type = genomic DNA
tgatcagttaactaaaagt

SEQ ID NO: 15 - DNA, 19 bp, Homo sapiens
  source 1..19: mol_type = genomic DNA
tttactagttcaagacaga

SEQ ID NO: 16 - DNA, 19 bp, Homo sapiens
  source 1..19: mol_type = genomic DNA
ctagttcaagacagatgaa

SEQ ID NO: 17 - DNA, 19 bp, Homo sapiens
  source 1..19: mol_type = genomic DNA
gaatgacccacgcaaaaaa

SEQ ID NO: 18 - DNA, 20 bp, synthetic construct
  source 1..20: mol_type = other DNA
ttgtacacgtggtcaagtgg

SEQ ID NO: 19 - DNA, 21 bp, synthetic construct
  source 1..21: mol_type = other DNA
tggagttgacattggtgaagg

SEQ ID NO: 20 - DNA, 20 bp, synthetic construct
  source 1..20: mol_type = other DNA
tgccattgccccatgtgaag

SEQ ID NO: 21 - DNA, 21 bp, synthetic construct
  source 1..21: mol_type = other DNA
agctgcacatcactcaggatt

SEQ ID NO: 22 - DNA, 19 bp, synthetic construct
  source 1..19: mol_type = other DNA
ctgagctcgccagtgaaat

SEQ ID NO: 23 - DNA, 19 bp, synthetic construct
  source 1..19: mol_type = other DNA
ctgtagtggtggtcggaga

SEQ ID NO: 24 - DNA, 20 bp, synthetic construct
  source 1..20: mol_type = other DNA
agatgcaataaccacccctg

SEQ ID NO: 25 - DNA, 20 bp, synthetic construct
  source 1..20: mol_type = other DNA
tgcgcagaatgagatgagtt

SEQ ID NO: 26 - DNA, 20 bp, synthetic construct
  source 1..20: mol_type = other DNA
ctgtagcccatgttgtagca

SEQ ID NO: 27 - DNA, 20 bp, synthetic construct
  source 1..20: mol_type = other DNA
ggttatctctcagctccacg

SEQ ID NO: 28 - DNA, 20 bp, synthetic construct
  source 1..20: mol_type = other DNA
gaatgtccaacgcaaagcaa

SEQ ID NO: 29 - DNA, 20 bp, synthetic construct
  source 1..20: mol_type = other DNA
acctcgaaacagcatctgac

SEQ ID NO: 30 - DNA, 20 bp, synthetic construct
  source 1..20: mol_type = other DNA
gtgtttgacggcatcccacc

SEQ ID NO: 31 - DNA, 20 bp, synthetic construct
  source 1..20: mol_type = other DNA
taggcttcagacgcacgacc

SEQ ID NO: 32 - RNA, 19 nt, synthetic construct
  source 1..19: mol_type = other RNA
  modified_base 16: mod_base = OTHER; note = Nucleotide with phosphorothioate linkage
  modified_base 17: mod_base = OTHER; note = Nucleotide with phosphorothioate linkage
  modified_base 18: mod_base = OTHER; note = Nucleotide with phosphorothioate linkage
ttggagccatccaaactct